(19) United States
(12) Patent Application Publication
(10) Pub. No.: US 2026/0055089 A1
(43) Pub. Date: Feb. 26, 2026

(54) NOVEL SUBSTITUTED BENZOTHIOPHENE-6-CARBOXYLIC ACID DERIVATIVES, PROCESSES FOR THEIR PREPARATION AND THERAPEUTIC USES THEREOF

(71) Applicant: SANOFI, Paris (FR)

(72) Inventors: Youssef El-Ahmad, Paris (FR); Frank Halley, Paris (FR)

(21) Appl. No.: 19/105,425

(22) PCT Filed: Aug. 24, 2023
(86) PCT No.: PCT/EP2023/073278

§ 371 Date: Feb. 21, 2025

(30) Foreign Application Priority Data
Aug. 25, 2022 (EP) 22306255.5

(51) Int. Cl. C07D 409/12; A61K 31/397; A61K 31/4025

(52) U.S. Cl. CPC C07D 409/12; A61K 31/397; A61K 31/4025

(57) ABSTRACT

Disclosed herein are compounds of the formula (I) or a pharmaceutically acceptable salt thereof (I) Wherein R1 and R2 independently represent a hydrogen atom or a deuterium atom; R3′ and R3″ independently represent a hydrogen atom, a methyl group, a methoxy group, a chlorine atom, a fluorine atom, or a cyano group; R4 represents a hydrogen atom or a fluorine atom; R5 and R5′ independently represent a hydrogen atom or a fluorine atom; Y represents —CH₂—, —CH═, —CR8=, —O— or —NH—, wherein R8 represents a fluorine atom or a (C₁-C₃)alkyl group; represents a single bond or a double bond; X represents —CH═, —N═ or —CR″═, wherein R″ represents a (C₁-C₃)alkyl group or a halogen atom, a cyano group, or a (C₁-C₃)fluoroalkyl group; R7 independently represents a (C1-C3)alkyl group, such as a methyl group, a halogen atom, or a (C1-C3)fluoroalkyl group, and R6 represents an (C₆-C₁₀)aryl group or a 5- or 6-membered heteroaryl group.

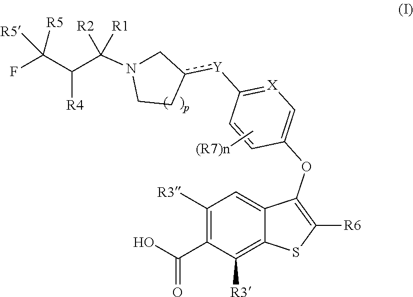

Disclosed herein are novel substituted benzothiophene-6-carboxylic acid derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3-antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use in cancer treatment.

Disclosed herein are compounds of the formula (I), or pharmaceutically acceptable salts thereof:

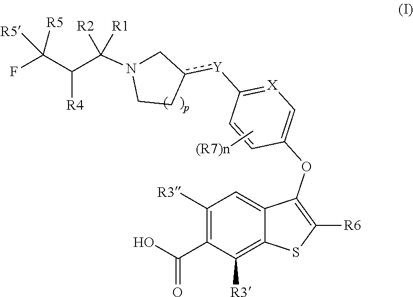

wherein:
R1 and R2 independently represent a hydrogen atom or a deuterium atom;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
Y represents —CH₂—, —CH═, —CR8=, —O— or —NH—, wherein R8 represents a fluorine atom or a (C₁-C₃)alkyl group;
[symbol] represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
n is 0, 1 or 2; and
R6 represents a (C₆-C₁₀)aryl group or a 5- or 6-membered heteroaryl group, said (C₆-C₁₀)aryl group or a 5- or 6-membered monocyclic heteroaryl group being optionally substituted with 1 to 3 groups selected from —Z—R6a and R6a;
wherein
Z is methylene;
R6a is selected from
   a —OH group,
   a (C₁-C₆)alkyl group optionally substituted by a cyano group or a —OH group,
   a (C₁-C₆)alkylene group,
   a halogen atom,
   a nitro group,
   a cyano group,
   a (C₁-C₆)fluoroalkyl group,
   a (C₁-C₆)fluoroalkoxy group,
   a (C₃-C₆)cycloalkyl group,
   a (C₁-C₆)alkoxy group,
   a —SF₅ group,
   a trifluoromethylsulfonyl group,
   a (C₁-C₄)alkylthio group;
   a (C₁-C₄)fluoroalkylthio group,
   a (C₁-C₄)alkylsulfonyl group
   a —NR9R10 group,
   a —C(O)R9 group,
   a (C₃-C₇)cycloalkyl group, and
   a 4-7 membered saturated, unsaturated or partially saturated ring containing one to four heteroatoms or groups selected from O, NH, C(O) and S(O)₀₋₂;
   wherein R9 and R10 are independently selected from a hydrogen atom and a (C₁-C₄)alkyl group; or R9 and R10 together with the nitrogen to which they are both attached form a 4 to 7 member saturated ring containing one other heteroatom or group selected from O, NH, and S(O)₀₋₂; wherein said 4-7 member ring can be unsubstituted or substituted with a (C₁-C₄)alkyl group.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an alkyl group: a linear or branched saturated hydrocarbon-based aliphatic group comprising, unless otherwise mentioned, from 1 to 6 carbon atoms (noted “(C1-C6)-alkyl”). By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
an alkylene group: a linear or branched hydrocarbon-based aliphatic group comprising, unless otherwise mentioned, from 1 to 6 carbon atoms (noted “(C₁-C₆)-alkylene”) and at least an unsaturation. By way of examples, mention may be made of, but not limited to: vinyl group, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of CH₂F, CHF₂, CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a 5- or 6-membered monocyclic heteroaryl group: a cyclic 5 or 6-membered aromatic group. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl;
a (C₆-C₁₀)aryl group: a monocyclic or fused bicyclic aromatic ring assembly containing six to ten ring carbon atoms.

In another embodiment, in the compounds of formula (I) as defined above, R1 and R2 are a hydrogen atom.

In another embodiment, in the compounds of formula (I) as defined above, R3′ and R3″ both represent a hydrogen atom.

In another embodiment, in the compounds of formula (I) as defined above, R4 is a hydrogen atom.

In another embodiment, in the compounds of formula (I) as defined above, R5 and R5′ both represent a hydrogen atom.

In another embodiment, in the compounds of formula (I) as defined above, p is 0. In another embodiment, in the compounds of formula (I) as defined above, p is 1.

In another embodiment, in the compounds of formula (I) as defined above, Y is —O— or —NH—.

In another embodiment, in the compounds of formula (I) as defined above, n is 0. In another embodiment, in the compounds of formula (I) as defined above, n is 1. In another embodiment, in the compounds of formula (I) as defined above, n is 2.

In another embodiment, in the compounds of formula (I) as defined above, X represents —CH═.

In another embodiment, in the compounds of formula (I) as defined above, R6 represents a phenyl group optionally substituted by 1, 2, 3 or 4 groups selected from a halogen atom, and a (C₁-C₆)fluoroalkyl group, more particularly optionally substituted by 2 or 3 groups selected from a halogen, for example a fluorine atom and a (C₁-C₄)fluoroalkyl group and even more particularly by one fluorine atom and by one difluoroethyl group, for example a 1,1-difluoroethyl group.

In another embodiment, in the compound of formula (I) as defined above, X represents —CH═, and R6 represents a phenyl group optionally substituted by 1, 2, 3 or 4 groups selected from a halogen atom, and a (C₁-C₆)fluoroalkyl group, more particularly optionally substituted by 2 or 3 groups selected from a halogen, for example a fluorine atom and a (C₁-C₄)fluoroalkyl group and even more particularly by one fluorine atom and by one difluoroethyl group, for example a 1,1-difluoroethyl group.

In an embodiment, X represents a carbon atom (i.e., a ═CH— group) in the compounds of formula (I). Accordingly, herein are described compounds of formula (II):

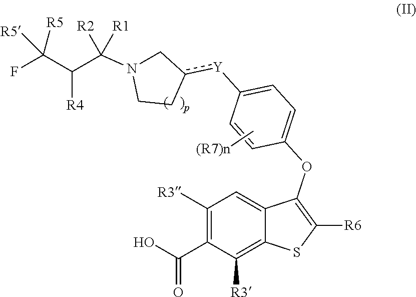

wherein R1, R2, R3′, R3″, R4, R5, R5′, R6, R7, [symbol] , Y, n and p are as defined in formula (II) hereabove.

The embodiments as described above for compounds of formula (I) are also suitable for compounds of formula (II).

In another embodiment, in the compounds of formula (II) as defined above, R6 represents a phenyl group optionally substituted by 1, 2, 3 or 4 groups selected from a halogen atom, and a (C₁-C₆)alkyl group, more particularly optionally substituted by 2, 3 or 4 groups selected from a halogen, for example a fluorine atom, and a methyl group and even more particularly by one fluorine atom and by two methyl groups.

Among the compounds of formula (I) described herein, mention may be made in particular of the following compounds or a pharmaceutically acceptable salt thereof, in particular hydrochloride salt thereof:
2-(2-(1,1-difluoroethyl)-4-fluorophenyl)-3-(4-((1-(3-fluoropropyl)azetidin-3-yl)amino)phenoxy)benzo[b]thiophene-6-carboxylic acid, hydrochloride (1),
2-(2-(1,1-difluoroethyl)-4-fluorophenyl)-3-(4-((1-(3-fluoropropyl)azetidin-3-yl)oxy)phenoxy)benzo[b]thiophene-6-carboxylic acid (2), and
(S)-2-(2-(1,1-difluoroethyl)-4-fluorophenyl)-3-(4-((1-(3-fluoropropyl)pyrrolidin-3-yl)oxy)phenoxy)benzo[b]thiophene-6-carboxylic acid (3).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) are described below.

The following abbreviations and empirical formulae are used:
CO Carbon monoxide
Cs₂CO₃ Cesium carbonate
DCM Dichloromethane
DMF N,N-dimethylformamide
DMSO Dimethyl sulfoxide
EtOH Ethanol
EtOAc Ethyl acetate
H₂ Hydrogen
H₂O₂ Hydrogen peroxide
HCl Hydrochloric acid
HPLC High performance liquid chromatography
LiOH Lithium hydroxide
MeOH Methanol
MgSO₄ Magnesium sulfate
N₂ Nitrogen
Pd/C Palladium on carbon
Pd(OH)₂ Palladium hydroxide
K₂CO₃ Potassium carbonate
NaHCO₃ Sodium bicarbonate
NaH Sodium hydride
NaOH Sodium hydroxide
NaI Sodium iodide
NaNO₂ Sodium nitrite
Na₂SO₄ Sodium sulfate
NaHSO₃ Sodium bisulfate
NBS N-bromosuccinimide
Pd(dppf)Cl₂ [1,1′-Bis(diphenylphosphino)ferrocene]dichloropalladium(II)
SNAr Aromatic Nucleophilic Substitution
TEA Triethylamine
TFA Trifluoroacetic acid
THF Tetrahydrofuran
TiCl₃ Titanium(III) chloride
RT Room temperature

### Scheme 1a Parts 1 and 2: Preparation of Compounds of the Formula (I)—General Process

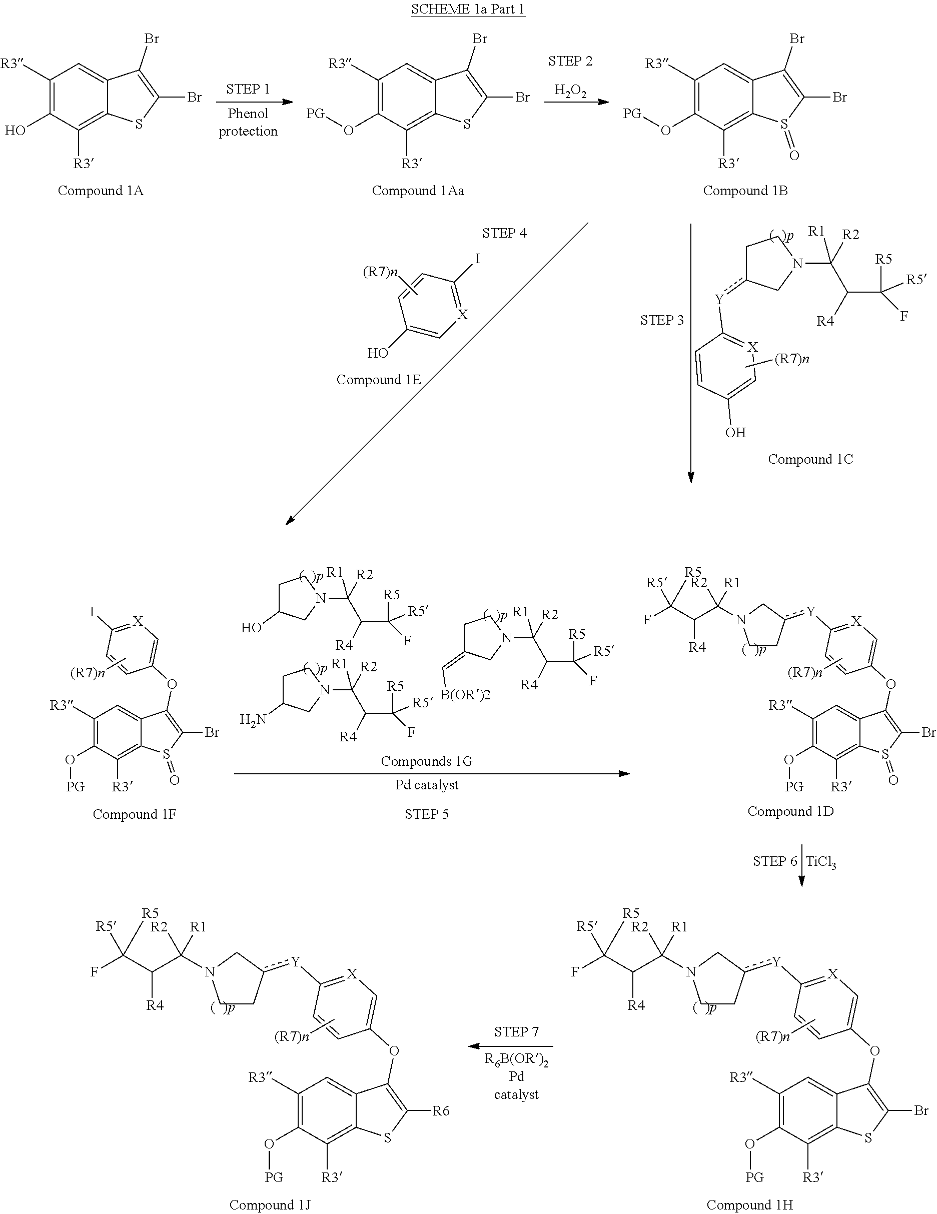

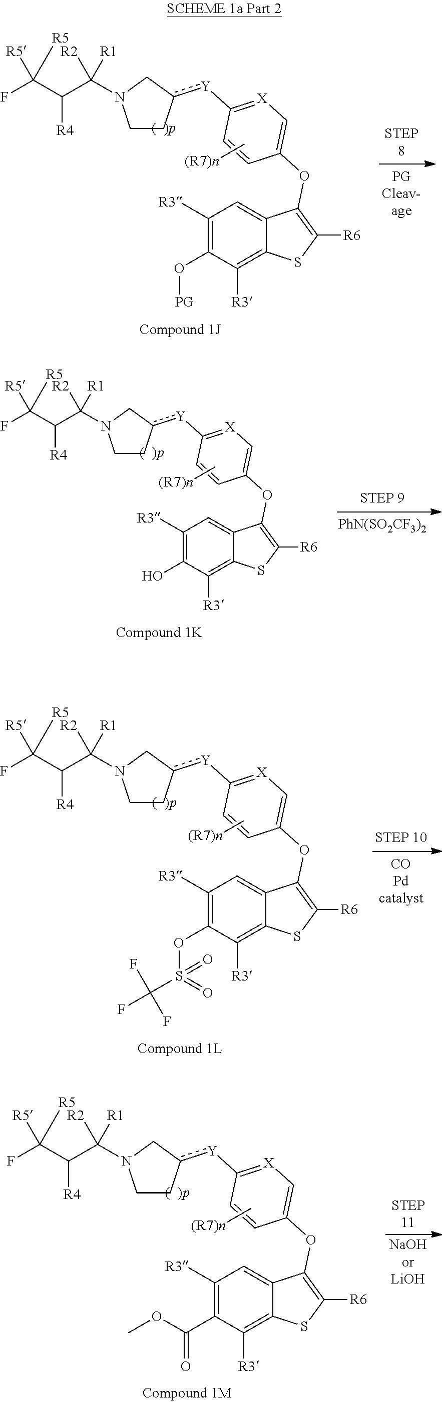

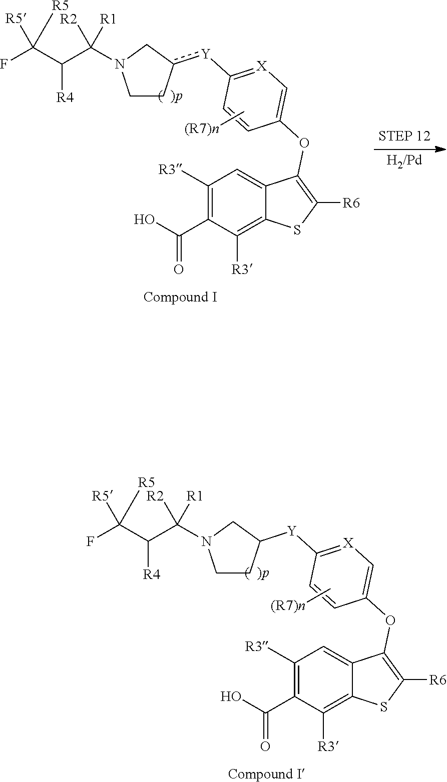

According to SCHEME 1a—Part-1 and Part-2, in which R1, R2, R3′, R3″, R4, R5, R5′, R6, R7, [symbol] , n, p, X and Y are defined as described above, compound 1A (prepared according to WO2004009086) can be converted in STEP 1 to compound 1Aa by protecting the phenol moiety by a protecting group (PG) such as pivaloyl, benzyl, para-methoxy-benzyl or 2,4-dimethoxy-benzyl.

Compound 1Aa can be converted in STEP 2 to compound 1B in oxidation conditions such as hydrogen peroxide (H₂O₂).

Compound 1B can be converted in STEP 3 to compound 1D by treatment with compound 1C in a SNAr reaction in a solvent such as DMF, in the presence of a base, for example cesium carbonate (Cs₂CO₃), by heating up to reflux of solvent.

Alternatively, compound 1D can be obtained by treatment of compound 1B with compound 1E in STEP 4 in a SNAr reaction in a solvent such as DMF, in the presence of a base, for example sodium hydride (NaH), by heating up to reflux of solvent, followed by coupling reaction in STEP 5 between obtained compound 1F and one of the compounds 1G in coupling reaction conditions.

Compound 1D can be converted in STEP 6 to compound 1H in reduction conditions such as TiCl₃.

Compound 1H can be converted to compound 1J in STEP 7 through a Suzuki coupling with a suitable boronic reagent R6B(OR′)₂, wherein —B(OR′)₂ is a boronic acid or a pinacolate ester and R6 is defined as above, using for example Pd(dppf)Cl₂, complex with DCM, as catalyst, in a mixture of dioxane and water as solvent and in the presence of a base, for example Cs₂CO₃, at RT or by heating up to reflux of the solvent.

Compound 1J can be converted to compound 1K in STEP 8 by cleaving the protecting group (PG) under conditions linked to the nature of PG.

Compound 1K can be converted in STEP 9 to compound 1L by treatment, for example with 1,1,1-trifluoro-N-phenyl-N-((trifluoromethyl)sulfonyl)methanesulfonamide (PhN(SO₂CF₃)₂, in solution in DCM, in the presence of pyridine as a base.

Compound 1L can be converted in STEP 10 to compound 1M by carbonylation with carbon monoxide, in solution in DMF and MeOH, in the presence of a palladium catalyst, for example [1,1′-bis(diphenylphosphino)ferrocene]dichloropalladium(II) (Pd(dppf)Cl₂), complex with DCM.

Compound 1M can be converted in STEP 11 to compound I, in presence of a source of hydroxide ions, in particular by treatment with an aqueous solution of sodium hydroxide (NaOH) 2N or lithium hydroxide (LiOH) in MeOH or THF. Extraction of the product could give the sodium or lithium salt of compound I. The acidification with an aqueous solution of HCl to pH 6-7 could give the neutral form of compound L The acidification with an aqueous solution of HCl to pH 1-2 could give the hydrochloride salt of compound I. The purification using HPLC in presence of formic acid or trifluoroacetic acid in the eluent could give the formate or trifluoroacetate salt of compound I.

When Y=—CH═, compound I may be reduced by hydrogenation in STEP 12 with a catalyst, such as Pd/C or Pd(OH)₂ under hydrogen (H₂) pressure, for example at temperature up to 70° C., to give the corresponding saturated compound I′.

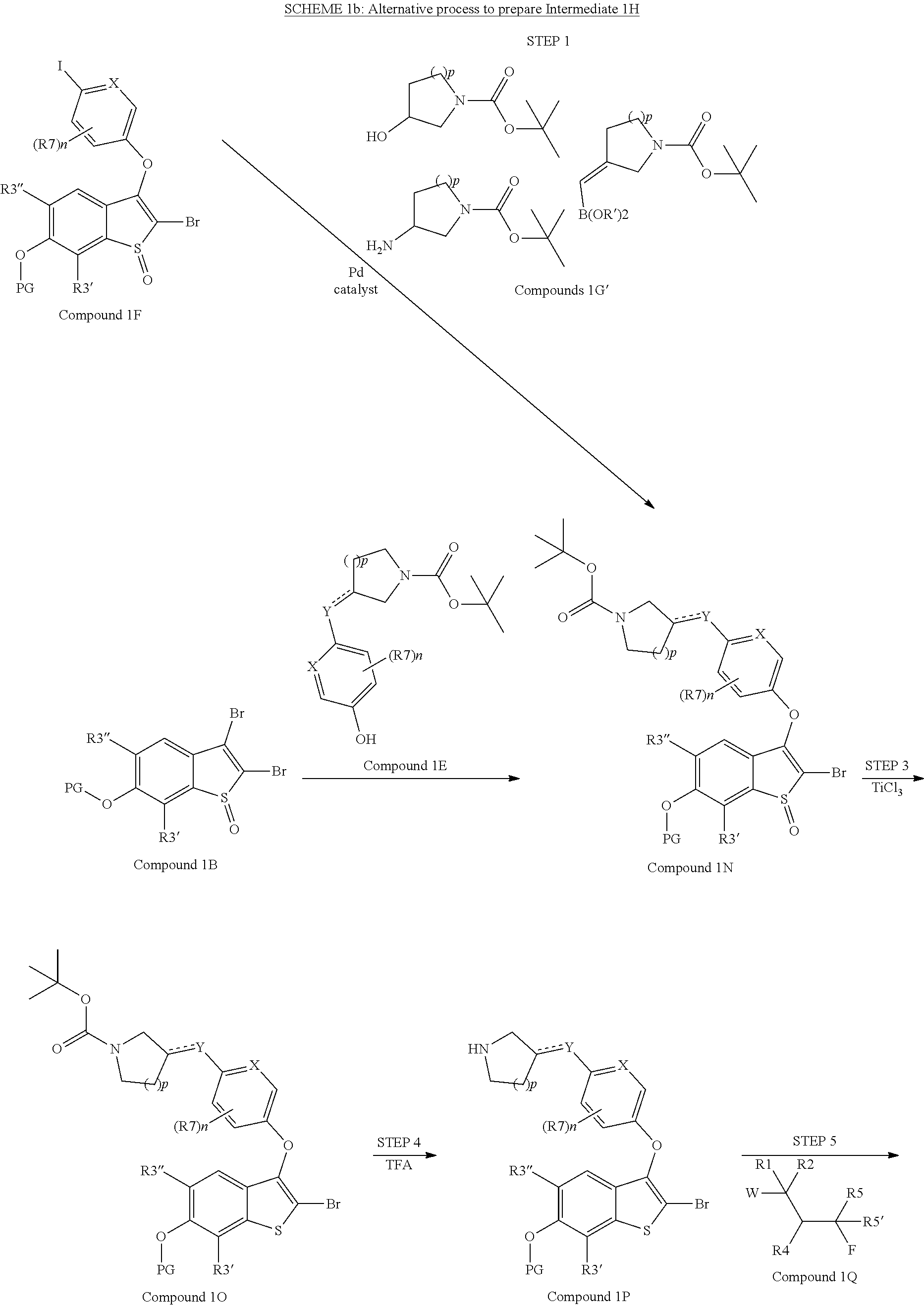

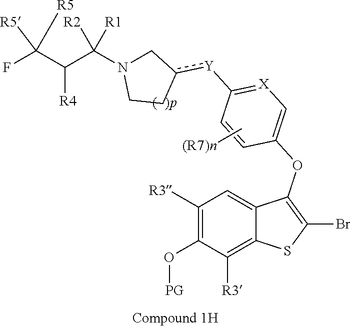

According to SCHEME 1b, in which R1, R2, R3′, R3″, R4, R5, R5′, R7, [symbol] , n, p, X and Y are defined as described above and PG is a protecting group such as pivaloyl, benzyl, para-methoxy-benzyl or 2,4-dimethoxy-benzyl, compound 1F can be converted in STEP 1 to compound 1N by reacting with one of the compounds 1G′ under coupling rection conditions.

Alternatively, compound 1N can be obtained in STEP 2 by treatment of compound 1B with compound 1E in a SNAr reaction in a solvent such as DMF, in the presence of a base, for example cesium carbonate (Cs₂CO₃), by heating up to reflux of solvent.

Compound 1N can be converted in STEP 3 to compound 10 in reduction conditions such as TiCl₃.

Compound 10 can be converted in STEP 4 to compound 1P by treatment with TFA or HCl.

Compound 1P can be converted to compound 1H in STEP 5 by the treatment with compound 1Q, wherein W is Cl, Br or I or OSO₂R with R=CH₃, PhMe, CF₃ or CF₂CF₂CF₂CF₃ in presence of a base such as potassium carbonate in DMF as a solvent.

### Scheme 1c Parts 1 and 2: Alternative Process to Prepare Intermediate 1M

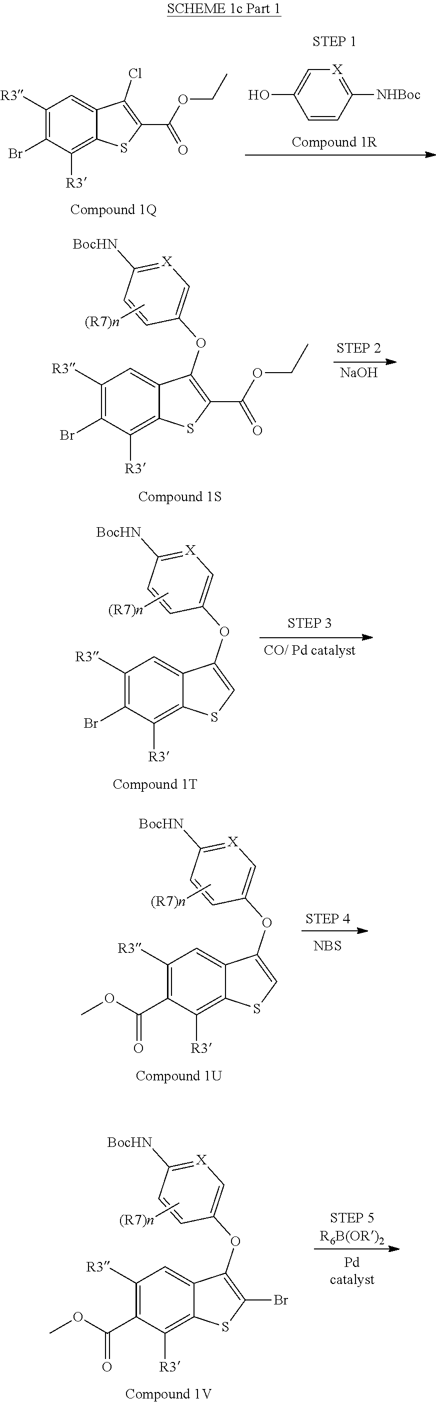

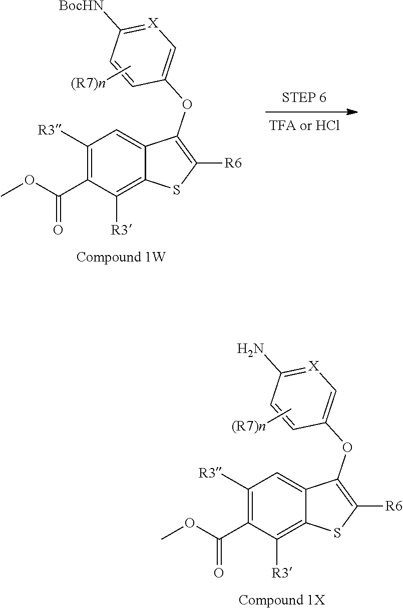

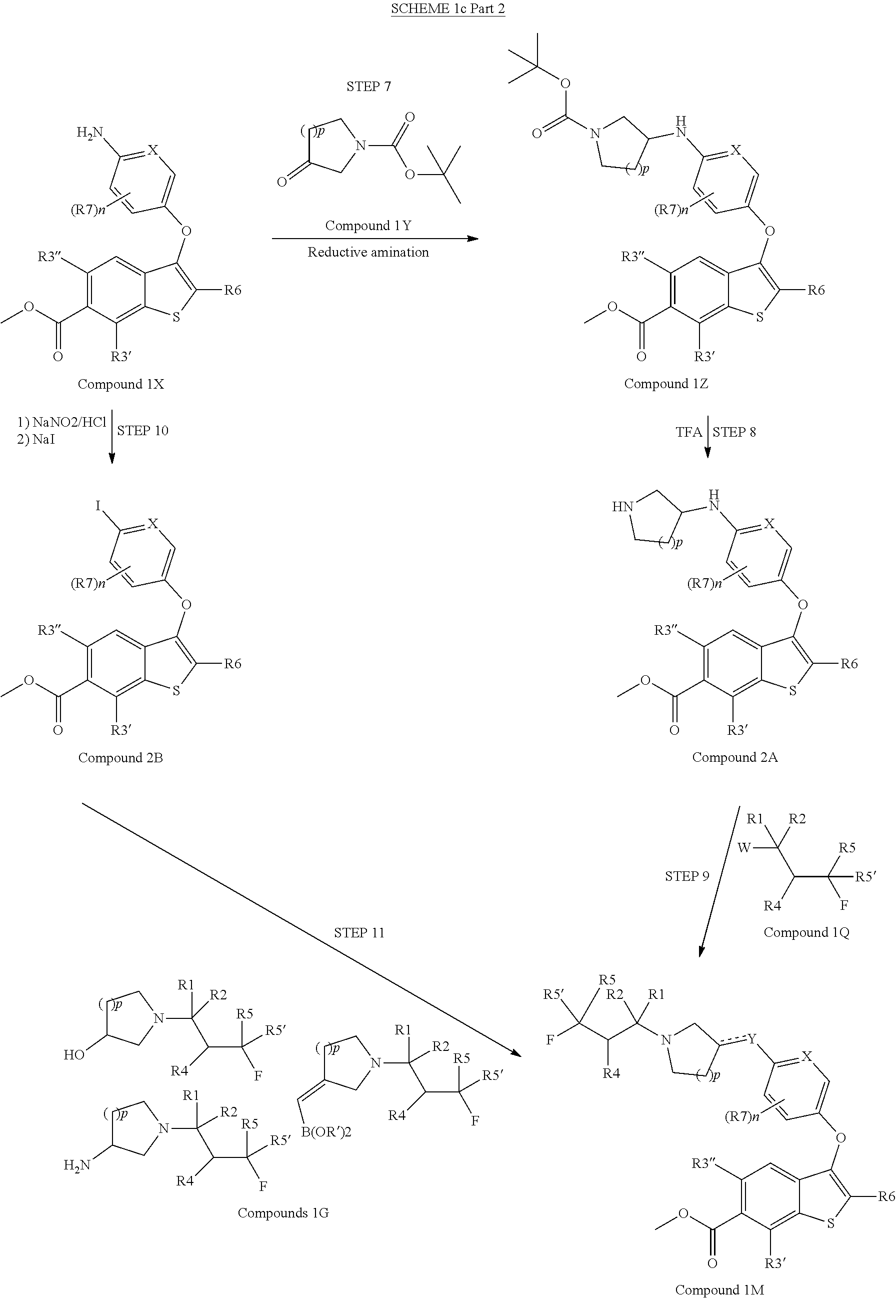

According to SCHEME 1c—Part-1 and Part-2, in which, R1, R2, R3′, R3″, R4, R5, R5′, R6, R7, [symbol] , n, p, X and Y are defined as described above, compound 1Q (prepared according to US20040009976) can be converted in STEP 1 to compound 1S by reacting with compound 1R in a SNAr reaction in a solvent such as DMF, in the presence of a base, for example NaOH, by heating up to reflux of solvent.

Compound 1S can be converted in STEP 2 to compound 1T in decarboxylation conditions by treatment with NaOH then heating up to 135° C.

Compound 1T can be converted in STEP 3 to compound 1U by carbonylation with carbon monoxide (CO), in solution in DMF and MeOH, in the presence of a palladium catalyst, for example [1,1′-bis(diphenylphosphino)ferrocene]dichloropalladium(II) (Pd(dppf)Cl₂), complex with DCM.

Compound 1U can be converted in STEP 4 to compound 1V by treatment with with a brominated reagent such as NBS.

Compound 1V can be converted to compound 1W in STEP 5 through a Suzuki coupling with a suitable boronic reagent R6B(OR′)₂, wherein —B(OR′)₂ is a boronic acid or a pinacolate ester and R6 is defined as above, using for example Pd(dppf)Cl₂, complex with DCM, as catalyst, in a mixture of dioxane and water as solvent and in the presence of a base, for example Cs₂CO₃, at RT or by heating up to reflux of the solvent.

Compound 1W can be converted in STEP 6 to compound 1X by treatment with TFA or HCl.

Compound 1X can be converted in STEP 7 to compound 1Z by treatment with compound 1Y in reductive amination conditions.

Compound 1Z can be converted in STEP 8 to compound 2A by treatment with TFA or HCl.

Compound 2A can be converted in STEP 9 to compound 1M by the treatment with compound 1Q, wherein W is Cl, Br or I or OSO₂R with R=CH₃, PhMe, CF₃ or CF₂CF₂CF₂CF₃ in presence of a base such as potassium carbonate in DMF as a solvent.

Alternatively, compound 1X can be converted to compound 2B in Sandmeyer conditions in STEP 10 by treatment with sodium nitrite (NaNO₂) followed by treatment with sodium (NaI).

Compound 2B can be converted to compound 1M in STEP 11 by coupling reaction with one of compounds 1G under coupling reaction conditions.

Herein is also provided a process for preparing a compound of formula (I) as defined above, wherein a compound of formula 1M

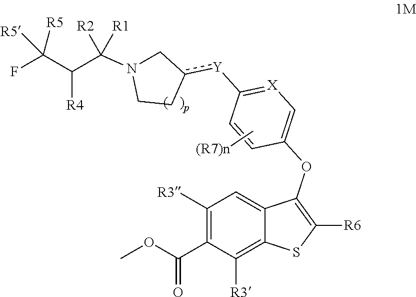

wherein R1, R2, R3′, R3″, R4, R5, R5′, R6, R7, [symbol] n, p, X and Y are defined as described above is converted to compound of formula (I), in presence of a source of hydroxide ions, such as NaOH or LiOH in solution in methanol or THF, said step being optionally preceded by a step of obtaining compound 1M, wherein a compound of formula 1L

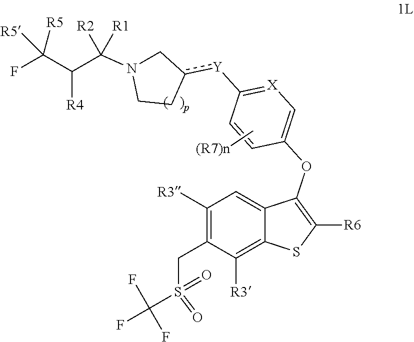

wherein R1, R2, R3′, R3″, R4, R5, R5′, R6, R7, [symbol] , n, p, X and Y are defined as described above, is converted to compound 1M by carbonylation with carbon monoxide, in solution in DMF and MeOH, in the presence of a palladium catalyst, with DCM.

Herein is also provided a process for preparing a compound of formula (I) as defined above, wherein a compound of formula 1M

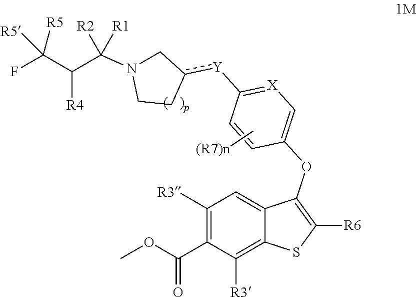

wherein R1, R2, R3′, R3″, R4, R5, R5′, R6, R7, [symbol] n, p, X and Y are defined as described above is converted to compound of formula (I), in presence of a source of hydroxide ions, such as NaOH or LiOH in solution in methanol or THF, said step being optionally preceded by a step of obtaining compound 1M, wherein a compound of formula 2B

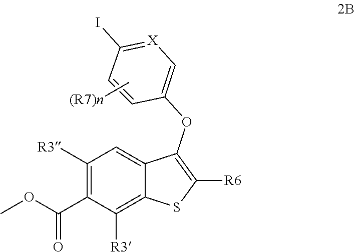

wherein R3′, R3″, R6, R7, n and X are defined as described above, is coupled with one of compounds 1G in coupling reaction conditions.

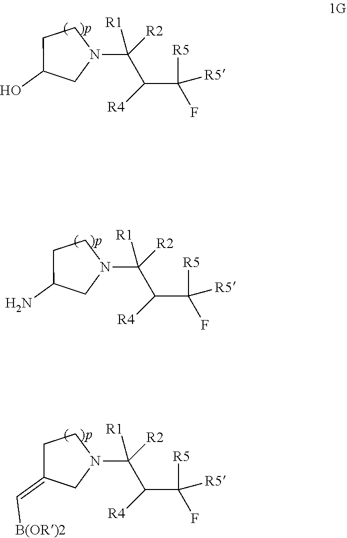

wherein R1, R2, R4, R5, R5′ and p are defined above.

Herein is also provided a process for preparing a compound of formula (I) as defined above, wherein a compound of formula 1M

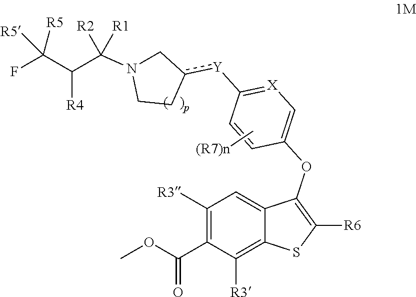

wherein R1, R2, R3′, R3″, R4, R5, R5′, R6, R7, [symbol] n, p, X and Y are defined as described above is converted to compound of formula (I), in presence of a source of hydroxide ions, such as NaOH or LiOH in solution in methanol or THF, said step being optionally preceded by a step of obtaining compound 1M, wherein a compound of formula 2A

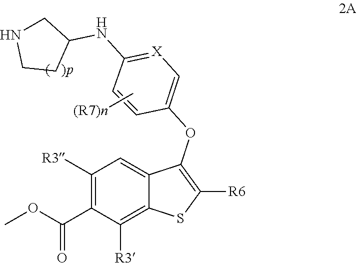

wherein R3′, R3″, R6, R7, n, p and X are defined as described above, is treated with compound 1Q

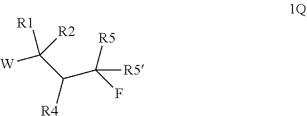

wherein W is Cl, Br or I or OSO₂R with R=CH₃, PhMe, CF₃ or CF₂CF₂CF₂CF₃ and R1, R2, R3′, R3″, R4, R5, R5′ and p are defined above, in presence of a base such as potassium carbonate in DMF as a solvent.

Herein are also provided the intermediate compounds selected from compounds 1M, 1H, 1L, 1X

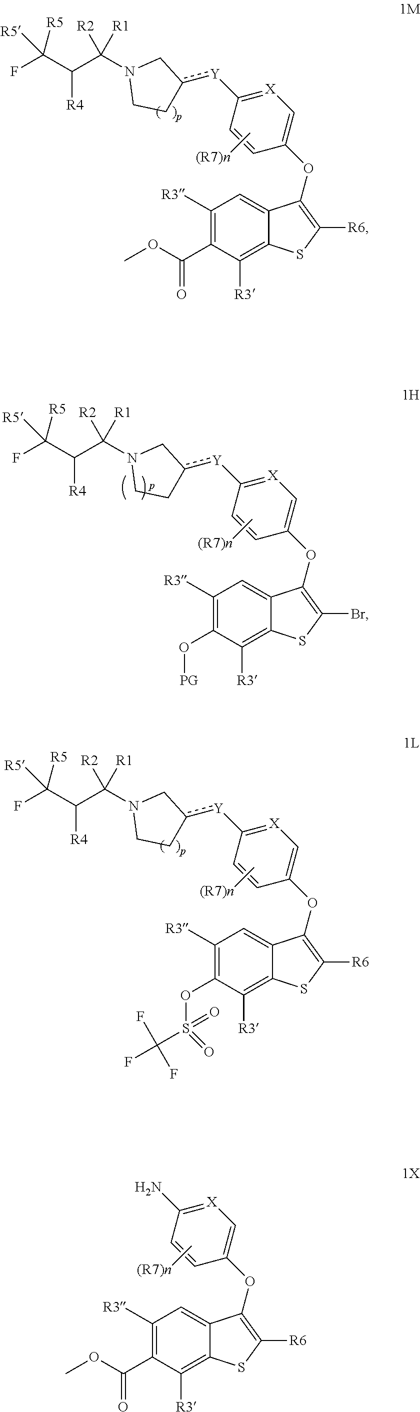

wherein R1, R2, R3, R3′, R3″, R4, R5, R5′, R6, R7, n, p, [symbol] X and Y are defined above and PG is a protecting group such as pivaloyl, benzyl, para-methoxy-benzyl or 2,4-dimethoxy-benzyl.

In another aspect, herein is also provided a process for the preparation of a compound of formula (I), comprising a deprotection step of a compound of formula 1M as defined above, optionally followed by a purification step.

Said purification step may for example consist, as illustrated in step 4 of example 1 herein after, in an acidification step, for example with an aqueous solution of hydrochloric acid.

The ¹H NMR Spectra at 400 and 500 MHz were performed on a Bruker Avance DRX-400 and Bruker Avance DPX-500 spectrometer, respectively, with the chemical shifts (δ in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprises respectively specific compounds of formula (I) (name and structure) in accordance with the present disclosure as well their characterization (¹H NMR and liquid chromatography/mass).

TABLE 1a
(the first column “Ex” corresponds to the compound and example
number)

| Ex | Structure | Name |
|---|---|---|
| 1 | 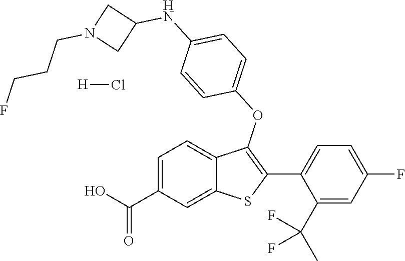 | 2-(2-(1,1-difluoroethyl)-4- fluorophenyl)-3-(4-((1-(3- fluoropropyl)azetidin-3- yl)amino)phenoxy)benzo[b] thiophene-6-carboxylic acid, hydrochloride |
| 2 | 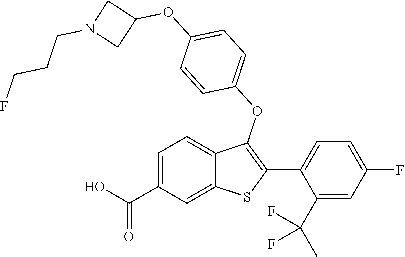 [SMILES: CC(F)(F)c1cc(F)ccc1-c1sc2cc(C(=O)O)ccc2c1Oc1ccc(OC2CN(CCCF)C2)cc1] | 2-(2-(1,1-difluoroethyl)-4- fluorophenyl)-3-(4-((1-(3- fluoropropyl)azetidin-3- yl)oxy)phenoxy)benzo[b] thiophene-6-carboxylic acid |
| 3 | 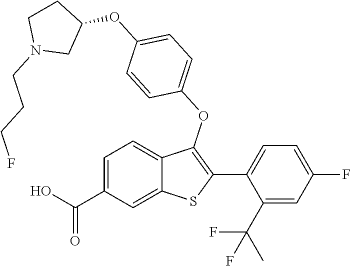 [SMILES: CC(F)(F)c1cc(F)ccc1-c1sc2cc(C(=O)O)ccc2c1Oc1ccc(O[C@H]2CCN(CCCF)C2)cc1] | (S)-2-(2-(1,1-difluoroethyl)-4- fluorophenyl)-3-(4-((1-(3- fluoropropyl)pyrrolidin-3- yl)oxy)phenoxy)benzo[b] thiophene-6-carboxylic acid |

TABLE 1b

| | | | MASS: |
|---|---|---|---|
| | Preparation | | LC/MS |
| Ex | Method | NMR | (m/z, MH+): |
| 1 | A | 1H-NMR (500 MHz, DMSO) δ 13.13 (s, 1H), 10.42 | 559 |
| | | (s, 1H), 8.64 (s, 1H), 7.89 (d, J = 9 Hz, 1H), 7.57- | |
| | | 7.50 (m, 2H), 7.42-7.38 (m, 2H), 6.73 (d, J = 9 Hz, | |
| | | 2H), 6.43 (d, J = 9 Hz, 2H), 6.13 (s, 1H), 4.53 (dt, | |
| | | J = 47, 6 Hz, 2H), 4.26-4.14 (m, 3H), 3.90-3.72 | |
| | | (m, 3H), 3.28 (s, 2H), 1.96 (t, J = 19 Hz, 3H), 1.89- | |
| | | 1.83 (m, 1H) | |
| 2 | B | 1H NMR (400 MHz, MeOD) δ 8.49 (s, 1H), 7.94 (d, | 560 |
| | | J = 8 Hz, 1H), 7.44-7.35 (m, 3H), 7.18-7.13 (m, | |
| | | 1H), 6.81-6.76 (m, 2H), 6.70-6.66 (m, 2H), 4.85- | |
| | | 4.81 (m, 1H), 4.55 (t, J = 6 Hz, 1H), 4.44 (t, J = 6 | |
| | | Hz, 1H), 4.16-4.10 (m, 2H), 3.67-3.61 (m, 2H), | |
| | | 3.01 (t, J = 8 Hz, 2H), 1.95-1.82 (m, 5H). | |
| 3 | B | 1H-NMR (400 MHz, MeOD) δ 8.47 (s, 1H), 7.93 | 574 |
| | | (d, J = 8 Hz, 1H), 7.48-7.32 (m, 3H), 7.15 (t, J = 7 | |
| | | Hz, 1H), 6.77 (s, 4H), 4.95 (s, 1H), 4.59 (t, J = 6 Hz, | |
| | | 1H), 4.47 (t, J = 6 Hz, 1H), 3.37 (d, J = 11 Hz, 2H), | |
| | | 3.29-3.23 (m, 1H), 3.17-3.05 (m, 3H), 2.38 (dd, | |
| | | J = 14, 6 Hz, 1H), 2.17-2.08 (m, 2H), 2.06-1.99 | |
| | | (m, 1H), 1.90 (t, J = 18.4 Hz, 3H) | |

The examples which follow describe the preparation of some compounds of formula (I) described herein. The numbers of the compounds exemplified below match those given in the Table 1 above. All reactions are performed under inert atmosphere, unless otherwise stated.

In the following examples, when the source of the starting products is not specified, it should be understood that said products are known compounds.

### EXAMPLES

### Intermediates

### Intermediate 1: Methyl 3-(4-aminophenoxy)-2-(2-(1,1-difluoroethyl)-4-fluorophenyl)benzo[b]thiophene-6-carboxylate

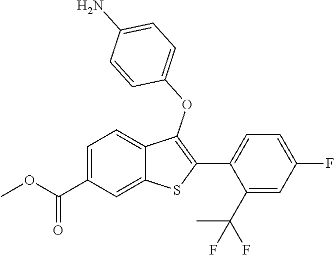 [SMILES: COC(=O)c1ccc2c(Oc3ccc(N)cc3)c(-c3ccc(F)cc3C(C)(F)F)sc2c1]

### Step 1: Ethyl 6-bromo-3-(4-((tert-butoxycarbonyl)amino)phenoxy)benzo[b]thiophene-2-carboxylate

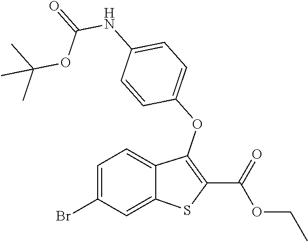 [SMILES: CCOC(=O)c1sc2cc(Br)ccc2c1Oc1ccc(NC(=O)OC(C)(C)C)cc1]

To a solution of ethyl 6-bromo-3-chlorobenzo[b]thiophene-2-carboxylate (prepared according to US20040009976) (5 g, 15.64 mmol) in DMSO (50 ml) were added tert-butyl N-(4-hydroxyphenyl)carbamate (3.93 g, 18.77 mmol) and K₂CO₃ (2.38 g, 17.21 mmol). The reaction was stirred at 100° C. for 18 h. The reaction mixture was cooled to RT, then water (10 ml) was added. The mixture was extracted with EtOAc (3×50 ml). The organic layers were dried over anhydrous Na₂SO₄, filtered and concentrated under reduced pressure. The residue obtained was purified by flash chromatography eluting with a gradient of cyclohexane/EtOAc from 100/00 to 70/30 to give 2.34 g (30%) of ethyl 6-bromo-3-(4-((tert-butoxycarbonyl)amino)phenoxy)benzo[b]thiophene-2-carboxylate as a yellow solid.

LC/MS (m/z, MH+): 492

### Step 2: Tert-butyl (4-((6-bromobenzo[b]thiophen-3-yl)oxy)phenyl)carbamate

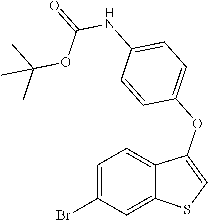 [SMILES: CC(C)(C)OC(=O)Nc1ccc(Oc2csc3cc(Br)ccc23)cc1]

NaOH in water (3M) (1.30 ml, 3.81 mmol) was added to a mixture of ethyl 6-bromo-3-(4-((tert-butoxycarbonyl)amino)phenoxy)benzo[b]thiophene-2-carboxylate (1.37 g, 2.78 mmol) in 1-butylpyrrolidin-2-one (10 ml) and water (0.2 ml). The mixture was heated at 65° C. for 1 h, then at 135° C. for 18 h. The reaction mixture was cooled down to RT then the mixture was diluted with EtOAc (20 ml) and water (20 ml). The organic layer was separated and washed with water again (2×20 ml) then dried over Na₂SO₄, filtered and concentrated under reduced pressure. The residue obtained was purified by flash chromatography eluting with a gradient of cyclohexane/EtOAc from 100/00 to 90/10 to give 587 mg (50%) of tert-butyl (4-((6-bromobenzo[b]thiophen-3-yl)oxy)phenyl)carbamate as a white solid. LC/MS (m/z, MH+): 420

### Step 3: Methyl 3-(4-((tert-butoxycarbonyl)amino)phenoxy)benzo[b]thiophene-6-carboxylate

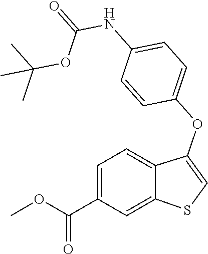 [SMILES: COC(=O)c1ccc2c(Oc3ccc(NC(=O)OC(C)(C)C)cc3)csc2c1]

In a 40 ml stainless steel bomb a mixture of tert-butyl (4-((6-bromobenzo[b]thiophen-3-yl)oxy)phenyl)carbamate (550 mg, 1.31 mmol), Et₃N (397 mg, 0.55 ml, 3.93 mmol) and [1,1′-bis(diphenylphosphino)ferrocene]dichloropalladium(II) complex with dichloromethane (214 mg, 0.26 mmol) in MeOH (12 ml) previously purged with N₂ was heated at 100° C. under CO atmosphere (5 bars) for 18 h. The reaction mixture was cooled to RT then evaporated under reduced pressure. The residue obtained was purified by flash chromatography eluting with cyclohexane/EtOAc from 100/00 to 90/10 to give 408 mg (78%) of methyl 3-(4-((tert-butoxycarbonyl)amino)phenoxy)benzo[b]thiophene-6-carboxylate as a white solid.

LC/MS (m/z, MH+): 400

### Step 4: Methyl 2-bromo-3-(4-((tert-butoxycarbonyl)amino)phenoxy)benzo[b]thiophene-6-carboxylate

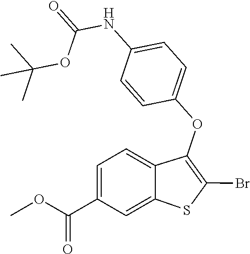 [SMILES: COC(=O)c1ccc2c(Oc3ccc(NC(=O)OC(C)(C)C)cc3)c(Br)sc2c1]

To a solution of methyl 3-(4-((tert-butoxycarbonyl)amino)phenoxy)benzo[b]thiophene-6-carboxylate (408 mg, 1.02 mmol) in DCM (10 ml) at RT was added NBS (191 mg, 1.07 mmol). The solution was stirred at RT for 18 h. A saturated aqueous solution of NaHCO₃ (20 ml) was added. The organic layer was separated, dried over anhydrous Na₂SO₄, filtered and concentrated under reduced pressure to give 460 mg (94%) of methyl 2-bromo-3-(4-((tert-butoxycarbonyl)amino)phenoxy)benzo[b]thiophene-6-carboxylate as a white solid. LC/MS (m/z, MH+): 478

### Step 5: Methyl 3-(4-((tert-butoxycarbonyl)amino)phenoxy)-2-(2-(1,1-difluoroethyl)-4-fluorophenyl)benzo[b]thiophene-6-carboxylate

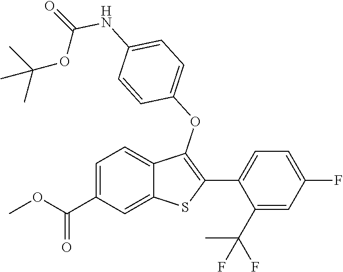 [SMILES: COC(=O)c1ccc2c(Oc3ccc(NC(=O)OC(C)(C)C)cc3)c(-c3ccc(F)cc3C(C)(F)F)sc2c1]

A mixture of methyl 2-bromo-3-(4-((tert-butoxycarbonyl)amino)phenoxy)benzo[b]thiophene-6-carboxylate (460 mg, 0.96 mmol), 2-[2-(1,1-difluoroethyl)-4-fluorophenyl]-4,4,5,5-tetramethyl-1,3,2-dioxaborolane (prepared according to J. Med. Chem., 2018, 61(7), 2837-2864) (590 mg, 1.44 mmol) and K₂CO₃ (266 mg, 1.92 mmol) in a mixture of dioxane (8 ml) and water (1.6 ml) was purged with N₂. [1,1′-Bis(diphenylphosphino)ferrocene]dichloropalladium(II) complex with dichloromethane (39 mg, 0.05 mmol) was added and the mixture was purged again with N₂ then heated to 80° C. for 18 h. After cooling down to RT, EtOAc (20 ml) and water (20 ml) were added. The aqueous layer was separated, extracted again with EtOAc (2×20 ml), dried over MgSO₄, filtered and evaporated under reduced pressure. The crude was purified by flash chromatography eluting with DCM to give 390 mg (72%) of methyl 3-(4-((tert-butoxycarbonyl)amino)phenoxy)-2-(2-(1,1-difluoroethyl)-4-fluorophenyl)benzo[b]thiophene-6-carboxylate as a white solid.

LC/MS (m/z, MH+): 558

### Step 6: Methyl 3-(4-aminophenoxy)-2-(2-(1,1-difluoroethyl)-4-fluorophenyl)benzo[b]thiophene-6-carboxylate

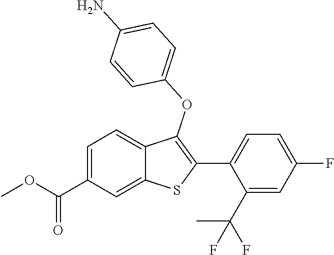 [SMILES: COC(=O)c1ccc2c(Oc3ccc(N)cc3)c(-c3ccc(F)cc3C(C)(F)F)sc2c1]

To a solution of methyl 3-(4-((tert-butoxycarbonyl)amino)phenoxy)-2-(2-(1,1-difluoroethyl)-4-fluorophenyl)benzo[b]thiophene-6-carboxylate (320 mg, 0.57 mmol) in HCl in dioxane (4M) (10 ml, 28.7 mmol). The reaction mixture was stirred at RT for 36 h. The reaction mixture was concentrated under reduced pressure and co-evaporated with Et₂O (2×10 ml). The crude was solubilized again in DCM (20 ml) and basified with an aqueous solution of NaHCO₃ and extracted with DCM (3×20 ml). The combined organic layers were dried over Na₂SO₄, filtered and concentrated under reduced pressure to give 224 mg (85%) of methyl 3-(4-aminophenoxy)-2-(2-(1,1-difluoroethyl)-4-fluorophenyl)benzo[b]thiophene-6-carboxylate.

LC/MS (m/z, MH+): 458

### Intermediate 2: 6-(Benzyloxy)-2,3-dibromobenzo[b]thiophene 1-oxide

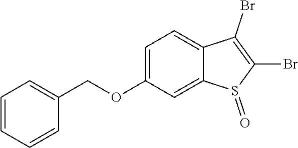 [SMILES: O=S1C(Br)=C(Br)c2ccc(OCc3ccccc3)cc21]

### Step 1: 6-(Benzyloxy)-2,3-dibromobenzo[b]thiophene

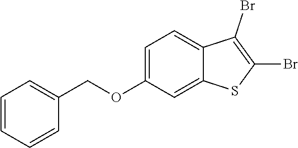 [SMILES: Brc1sc2cc(OCc3ccccc3)ccc2c1Br]

A solution of 2,3-dibromobenzothiophen-6-ol (prepared according to WO2004009086) (6.2 g, 20.1 mmol) in DMF (100 ml) was dropwise added to a suspension of NaH 60% (1.2 g, 30.2 mmol) in DMF (200 ml) at 0° C. After 20 minutes of stirring, benzyl bromide (6.89 g, 40.3 mmol) was added. The ice bath was removed. The reaction mixture was stirred at RT for 2 hours. The reaction mixture was poured into water (11). The solid formed was filtered and dried to give 8 g (99%) of 6-(benzyloxy)-2,3-dibromobenzo[b]thiophene.

LC/MS (m/z, MH+): 397

### Step 2: 6-(Benzyloxy)-2,3-dibromobenzo[b]thiophene 1-oxide

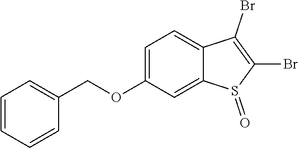 [SMILES: O=S1C(Br)=C(Br)c2ccc(OCc3ccccc3)cc21]

To the mixture of 6-(benzyloxy)-2,3-dibromobenzo[b]thiophene (6 g, 15.1 mmol) in DCM (30 ml) was added TFA (30 ml). The reaction mixture was stirred for 10 minutes at RT, then 30% aqueous solution of H₂O₂ (2.05 g, 18.1 mmol) was added. The reaction mixture was stirred for 4 hours at RT. NaHSO₃ (0.6 g) and water (30 ml) were added. The reaction mixture was stirred for 15 minutes and then concentrated under reduced pressure. To the residue obtained, DCM (100 ml) and saturated solution of NaHCO₃ (100 ml) were added. After decantation, the organic phase was dried over Na₂SO₄, filtered, and concentrated under reduced pressure. The residue obtained was purified by flash chromatography eluting with DCM to give 3 g (48%) of 6-(benzyloxy)-2,3-dibromobenzo[b]thiophene 1-oxide.

LC/MS (m/z, MH+): 413

### Examples

### Method A

### Example 1: 2-(2-(1,1-Difluoroethyl)-4-fluorophenyl)-3-(4-((1-(3-fluoropropyl)azetidin-3-yl)amino)phenoxy)benzo[b]thiophene-6-carboxylic acid, hydrochloride

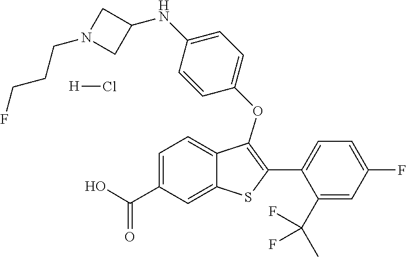

### Step 1: Tert-butyl 3-((4-((2-(2-(1,1-difluoroethyl)-4-fluorophenyl)-6-(methoxycarbonyl)benzo[b]thiophen-3-yl)oxy)phenyl)amino)azetidine-1-carboxylate

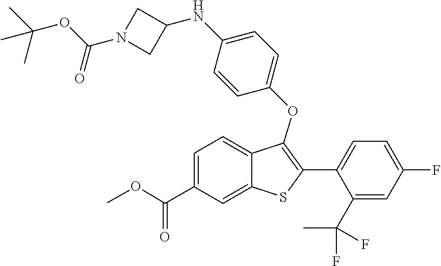 [SMILES: COC(=O)c1ccc2c(Oc3ccc(NC4CN(C(=O)OC(C)(C)C)C4)cc3)c(-c3ccc(F)cc3C(C)(F)F)sc2c1]

To a solution of tert-butyl 3-oxoazetidine-1-carboxylate (130 mg, 0.76 mmol) in EtOH (5 ml) were added methyl 3-(4-aminophenoxy)-2-(2-(1,1-difluoroethyl)-4-fluorophenyl)benzo[b]thiophene-6-carboxylate (Intermediate 1) (174 mg, 0.38 mmol) and acetic acid (44 μl, 0.76 mmol). The reaction mixture was stirred at RT for 30 min, then sodium cyanoborohydride (72 mg, 1.14 mmol) was added and the reaction mixture was stirred at 60° C. for 96 h. The reaction mixture was cooled to RT then concentrated under reduced pressure. EtOAc (20 ml) and water (20 ml) were added. After decantation, the organic phase dried over MgSO₄, filtered and concentrated under reduced pressure. The crude was purified by flash column chromatography eluting with a gradient of cyclohexane/EtOAc from 90/10 to 60/40 to give 111 mg (47%) of tert-butyl 3-((4-((2-(2-(1,1-difluoroethyl)-4-fluorophenyl)-6-(methoxycarbonyl)benzo[b]thiophen-3-yl)oxy)phenyl)amino)azetidine-1-carboxylate as a yellow solid.

LC/MS (m/z, MH+): 613

### Step 2: Methyl 3-(4-(azetidin-3-ylamino)phenoxy)-2-(2-(1,1-difluoroethyl)-4-fluorophenyl)benzo[b]thiophene-6-carboxylate

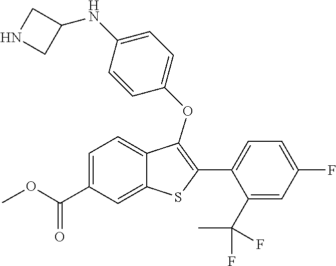 [SMILES: COC(=O)c1ccc2c(Oc3ccc(NC4CNC4)cc3)c(-c3ccc(F)cc3C(C)(F)F)sc2c1]

To a solution of tert-butyl 3-((4-((2-(2-(1,1-difluoroethyl)-4-fluorophenyl)-6-(methoxycarbonyl)benzo[b]thiophen-3-yl)oxy)phenyl)amino)azetidine-1-carboxylate (100 mg, 0.16 mmol) in DCM (2 ml) at RT was added TFA (0.24 ml, 3.26 mmol). The reaction mixture was stirred at RT for 12 h. The mixture was basified with an aqueous saturated solution of NaHCO₃ pH=8-9 and extracted with DCM (3×5 ml). The combined organic layers were dried over Na₂SO₄, filtered and concentrated under reduced pressure to give 62 mg (74%) of methyl 3-(4-(azetidin-3-ylamino)phenoxy)-2-(2-(1,1-difluoroethyl)-4-fluorophenyl)benzo[b]thiophene-6-carboxylate as a white solid.

LC/MS (m/z, MH+): 513

### Step 3: Methyl 2-(2-(1,1-difluoroethyl)-4-fluorophenyl)-3-(4-((1-(3-fluoropropyl)azetidin-3-yl)amino)phenoxy)benzo[b]thiophene-6-carboxylate

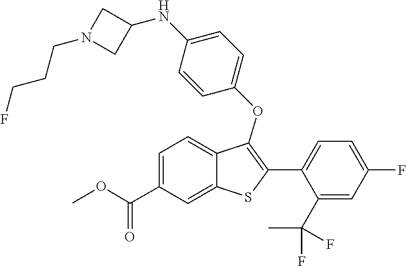 [SMILES: COC(=O)c1ccc2c(Oc3ccc(NC4CN(CCCF)C4)cc3)c(-c3ccc(F)cc3C(C)(F)F)sc2c1]

A mixture of methyl 3-(4-(azetidin-3-ylamino)phenoxy)-2-(2-(1,1-difluoroethyl)-4-fluorophenyl)benzo[b]thiophene-6-carboxylate (50 mg, 0.098 mmol), 1-fluoro-3-iodopropane (20 mg, 0.11 mmol) and K₂CO₃ (34 mg, 0.24 mmol) in a THF (1 ml) and water (0.1 ml) was stirred at 50° C. for 2.5 h. The mixture was cooled down to RT, water (10 ml) and EtOAc (20 ml) were added. After decantation, the organic phase was dried over MgSO₄, filtered, and evaporated reduced pressure. The residue obtained was purified by flash chromatography eluting with a gradient of DCM/isopropanol from (100/00) to (80/20) to give 37 mg (66%) of methyl 2-(2-(1,1-difluoroethyl)-4-fluorophenyl)-3-(4-((1-(3-fluoropropyl)azetidin-3-yl)amino)phenoxy)benzo[b]thiophene-6-carboxylate as a yellow solid.

LC/MS (m/z, MH+): 573

### Step 4: 2-(2-(1,1-Difluoroethyl)-4-fluorophenyl)-3-(4-((1-(3-fluoropropyl)azetidin-3-yl)amino)phenoxy)benzo[b]thiophene-6-carboxylic acid, hydrochloride

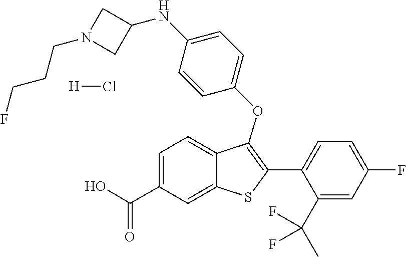

To a suspension of methyl 2-(2-(1,1-difluoroethyl)-4-fluorophenyl)-3-(4-((1-(3-fluoropropyl)azetidin-3-yl)amino)phenoxy)benzo[b]thiophene-6-carboxylate (40 mg, 0.07 mmol) in a mixture of water (0.40 ml) and THF (0.40 ml) was added LiOH·H₂O (15 mg, 0.35 mmol). The mixture was stirred at 50° C. for 10 hours. The reaction mixture was cooled down to RT then acidified with an aqueous solution of HCl (1N) until pH=2. The mixture was extracted with EtOAc (3×5 ml). The organic layers were washed with water (5 ml), dried over anhydrous Na₂SO₄, filtered and concentrated under reduced pressure. The crude was triturated with DCM (3×1 ml) and collected by centrifugation (3500 rpm, 15 min) to give 14 mg (36%) of 2-(2-(1,1-difluoroethyl)-4-fluorophenyl)-3-(4-((1-(3-fluoropropyl)azetidin-3-yl)amino)phenoxy)benzo[b]thiophene-6-carboxylic acid, hydrochloride as a yellow solid.

### Method B

### Example 2: 2-(2-(1,1-Difluoroethyl)-4-fluorophenyl)-3-(4-((1-(3-fluoropropyl)azetidin-3-yl)oxy)phenoxy)benzo[b]thiophene-6-carboxylic acid

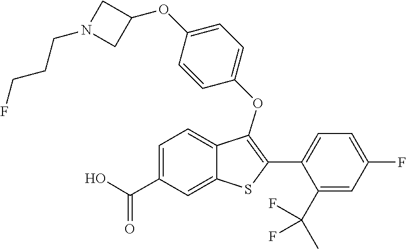 [SMILES: CC(F)(F)c1cc(F)ccc1-c1sc2cc(C(=O)O)ccc2c1Oc1ccc(OC2CN(CCCF)C2)cc1]

### Step 1: 6-(Benzyloxy)-2-bromo-3-(4-((1-(3-fluoropropyl)azetidin-3-yl)oxy)phenoxy)benzo[b]thiophene 1-oxide

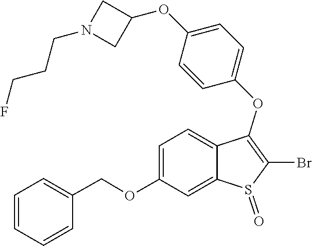 [SMILES: O=S1C(Br)=C(Oc2ccc(OC3CN(CCCF)C3)cc2)c2ccc(OCc3ccccc3)cc21]

To a solution of 6-(benzyloxy)-2,3-dibromobenzo[b]thiophene 1-oxide (Intermediate 2) (880 mg, 2.13 mmol) and 4-[1-(3-fluoropropyl)azetidin-3-yl]oxyphenol (prepared according to WO2019157020) (479 mg, 2.13 mmol) in N-methyl pyrrolidone (25 ml) was added Cs₂CO₃ (1.38 g, 4.25 mmol). The reaction mixture was stirred at 60° C. for 3 hours. After cooling to RT, water (10 ml) and EtOAc (20 ml) were added. After decantation, the organic phase was dried over MgSO₄, filtered and concentrated under reduced pressure. The residue obtained was purified by flash chromatography eluting with a gradient of petroleum ether/EtOAc from 100/00 to 00/100 to give 1 g (84%) of 6-(benzyloxy)-2-bromo-3-(4-((1-(3-fluoropropyl)azetidin-3-yl)oxy)phenoxy)benzo[b]thiophene 1-oxide as a brown solid. LC/MS (m/z, MH+): 558

### Step 2: 3-(4-((6-(Benzyloxy)-2-bromobenzo[b]thiophen-3-yl)oxy)phenoxy)-1-(3-fluoropropyl)azetidine

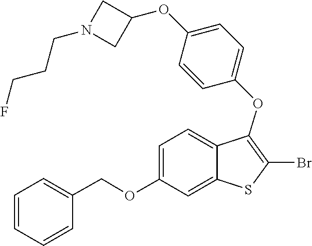 [SMILES: FCCCN1CC(Oc2ccc(Oc3c(Br)sc4cc(OCc5ccccc5)ccc34)cc2)C1]

To a solution of 6-(benzyloxy)-2-bromo-3-(4-((1-(3-fluoropropyl)azetidin-3-yl)oxy)phenoxy)benzo[b]thiophene 1-oxide (0.22 g, 0.39 mmol) in MeOH (8 ml) and chloroform (4 ml) was added a 15% solution of TiCl₃ in aqueous HCl (0.624 g, 0.788 mmol). The reaction mixture was stirred at RT for 2 hours. The reaction mixture was quenched with an aqueous saturated solution of NaHCO₃. After addition of DCM (50 ml) and decantation, the organic phase was washed with brine, dried over MgSO₄, filtered and concentrated under reduced pressure. The residue obtained was purified by flash chromatography eluting with a gradient of DCM/MeOH from 100/00 to 90/10 to give 190 mg (89%) of 3-(4-((6-(benzyloxy)-2-bromobenzo[b]thiophen-3-yl)oxy)phenoxy)-1-(3-fluoropropyl)azetidine as a brown solid.

LC/MS (m/z, MH+): 542

### Step 3: 3-(4-((6-(Benzyloxy)-2-(2-(1,1-difluoroethyl)-4-fluorophenyl)benzo[b]thiophen-3-yl)oxy)phenoxy)-1-(3-fluoropropyl)azetidine

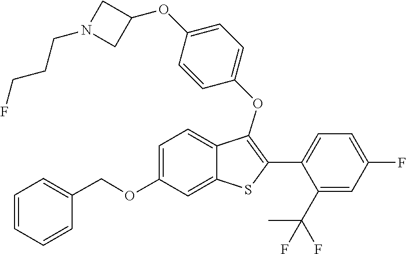 [SMILES: CC(F)(F)c1cc(F)ccc1-c1sc2cc(OCc3ccccc3)ccc2c1Oc1ccc(OC2CN(CCCF)C2)cc1]

To a solution of 3-(4-((6-(benzyloxy)-2-bromobenzo[b]thiophen-3-yl)oxy)phenoxy)-1-(3-fluoropropyl)azetidine (190 mg, 0.35 mmol) and 2-[2-(1,1-difluoroethyl)-4-fluoro-phenyl]-4,4,5,5-tetramethyl-1,3,2-dioxaborolane (prepared according to J. Med. Chem., 2018, 61(7), 2837-2864) (110 mg, 0.385 mmol) in dioxane (16 ml) and water (4 ml) was added Pd(dppf)Cl2 (51 mg, 0.07 mmol) and Cs₂CO₃ (342 mg, 1.05 mmol). The reaction mixture was stirred at 100° C. for 16 hours. Water (10 ml) and DCM (20 ml) were added. After decantation, the organic phase was dried over MgSO₄, filtered, and concentrated under reduced pressure. The residue obtained was purified by flash chromatography eluting with a gradient of petroleum ether/EtOAc from 100/00 to 75/25 to give 200 mg (92%) of 3-(4-((6-(benzyloxy)-2-(2-(1,1-difluoroethyl)-4-fluorophenyl)benzo[b]thiophen-3-yl)oxy)phenoxy)-1-(3-fluoropropyl)azetidine.

LC/MS (m/z, MH+): 622

### Step 4: 2-(2-(1,1-Difluoroethyl)-4-fluorophenyl)-3-(4-((1-(3-fluoropropyl)azetidin-3-yl)oxy)phenoxy)benzo[b]thiophen-6-ol

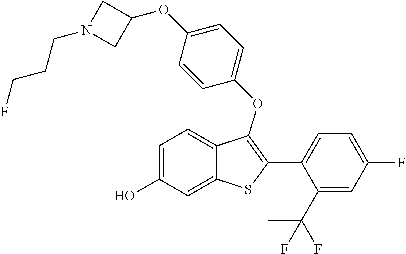 [SMILES: CC(F)(F)c1cc(F)ccc1-c1sc2cc(O)ccc2c1Oc1ccc(OC2CN(CCCF)C2)cc1]

A mixture of 3-(4-((6-(benzyloxy)-2-(2-(1,1-difluoroethyl)-4-fluorophenyl)benzo[b]thiophen-3-yl)oxy)phenoxy)-1-(3-fluoropropyl)azetidine (517 mg, 0.83 mmol), Pd/C (10%) (250 mg) and Pd(OH)₂/C (250 mg) in MeOH (20 ml) and EtOAc (20 ml) was stirred under H₂ (1 bar) at RT for 16 h. The reaction mixture was filtered, and the filtrate was concentrated under reduced pressure. The residue obtained was purified by HPLC (eluting with MeOH in basic water (0.05% NH₄HCO₃) from 78 to 82%) to give 362 mg (82%) of 2-(2-(1,1-difluoroethyl)-4-fluorophenyl)-3-(4-((1-(3-fluoropropyl)azetidin-3-yl)oxy)phenoxy)benzo[b]thiophen-6-ol.

LC/MS (m/z, MH+): 532

### Step 5: 2-(2-(1,1-Difluoroethyl)-4-fluorophenyl)-3-(4-((1-(3-fluoropropyl)azetidin-3-yl)oxy)phenoxy)benzo[b]thiophen-6-yl trifluoromethanesulfonate

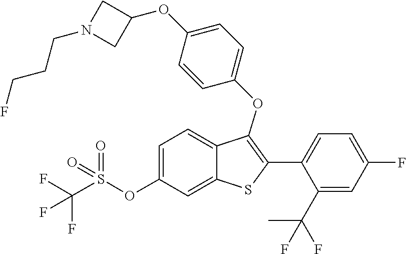 [SMILES: CC(F)(F)c1cc(F)ccc1-c1sc2cc(OS(=O)(=O)C(F)(F)F)ccc2c1Oc1ccc(OC2CN(CCCF)C2)cc1]

To a stirred mixture of 2-(2-(1,1-difluoroethyl)-4-fluorophenyl)-3-(4-((1-(3-fluoropropyl)azetidin-3-yl)oxy)phenoxy)benzo[b]thiophen-6-ol (30 mg, 0.056 mmol) and Et₃N (17 mg, 0.17 mmol) in DCM (10 ml) was added 1,1,1-trifluoro-N-phenyl-N-((trifluoromethyl)sulfonyl)methanesulfonamide (30 mg, 0.084 mmol) at RT. The reaction mixture was stirred at RT for 16 h. The reaction mixture was partitioned between DCM (10 ml) and water (5 ml). After decantation, the organic phase was dried over MgSO₄ and concentrated under reduced pressure. The residue obtained was purified by flash chromatography eluting with a gradient of petroleum ether/EtOAc from 65/35 to 60/40 to give 30 mg (80%) of 2-(2-(1,1-difluoroethyl)-4-fluorophenyl)-3-(4-((1-(3-fluoropropyl)azetidin-3-yl)oxy)phenoxy)benzo[b]thiophen-6-yl trifluoromethanesulfonate as a yellow oil.

LC/MS (m/z, MH+): 664

### Step 6: Methyl 2-(2-(1,1-difluoroethyl)-4-fluorophenyl)-3-(4-((1-(3-fluoropropyl)azetidin-3-yl)oxy)phenoxy)benzo[b]thiophene-6-carboxylate

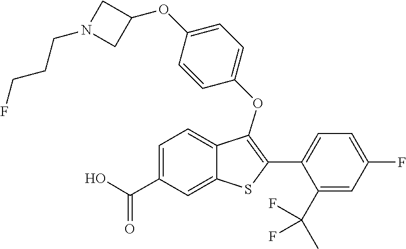 [SMILES: CC(F)(F)c1cc(F)ccc1-c1sc2cc(C(=O)O)ccc2c1Oc1ccc(OC2CN(CCCF)C2)cc1]

To a solution of 2-(2-(1,1-difluoroethyl)-4-fluorophenyl)-3-(4-((1-(3-fluoropropyl)azetidin-3-yl)oxy)phenoxy)benzo[b]thiophen-6-yl trifluoromethanesulfonate (110 mg. 0.166 mmol) in MeOH (10 ml) were added Pd(dppf)Cl₂ (18 mg, 0.025 mmol) and N-ethyl-N-isopropyl-propan-2-amine (214 mg, 1.66 mmol). The reaction mixture was stirred under 1 bar of CO at 68° C. for 1 h. The reaction mixture was filtered then the filtrate was concentrated under reduced pressure. The residue was partitioned between EtOAc (10 ml) and water (5 ml). After decantation, the organic phase was dried over MgSO₄, and concentrated under reduced pressure. The residue obtained was purified by flash chromatography eluting with a gradient of petroleum ether/EtOAc from 95/5 to 85/15 to give 90 mg (95%) of methyl 2-(2-(1,1-difluoroethyl)-4-fluorophenyl)-3-(4-((1-(3-fluoropropyl)azetidin-3-yl)oxy)phenoxy)benzo[b]thiophene-6-carboxylate.

LC/MS (m/z, MH+): 574

### Step 7: 2-(2-(1,1-Difluoroethyl)-4-fluorophenyl)-3-(4-((1-(3-fluoropropyl)azetidin-3-yl)oxy)phenoxy)benzo[b]thiophene-6-carboxylic acid

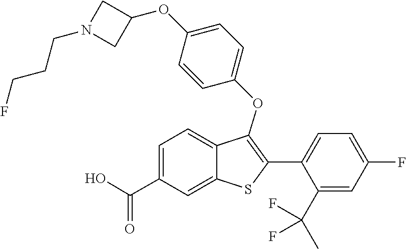 [SMILES: CC(F)(F)c1cc(F)ccc1-c1sc2cc(C(=O)O)ccc2c1Oc1ccc(OC2CN(CCCF)C2)cc1]

Step 7 of Example 2 was prepared following a similar procedure to that of step 4 of Example 1 from methyl 2-(2-(1,1-difluoroethyl)-4-fluorophenyl)-3-(4-((1-(3-fluoropropyl)azetidin-3-yl)oxy)phenoxy)benzo[b]thiophene-6-carboxylate to give 30 mg (56%) of 2-(2-(1,1-difluoroethyl)-4-fluorophenyl)-3-(4-((1-(3-fluoropropyl)azetidin-3-yl)oxy)phenoxy)benzo[b]thiophene-6-carboxylic acid as a white solid.

### Example 3: (S)-2-(2-(1,1-Difluoroethyl)-4-fluorophenyl)-3-(4-((1-(3-fluoropropyl)pyrrolidin-3-yl)oxy)phenoxy)benzo[b]thiophene-6-carboxylic acid

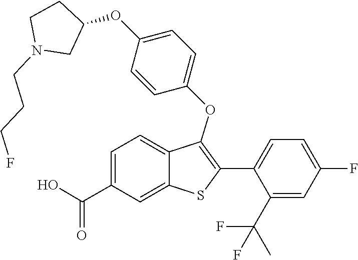 [SMILES: CC(F)(F)c1cc(F)ccc1-c1sc2cc(C(=O)O)ccc2c1Oc1ccc(O[C@H]2CCN(CCCF)C2)cc1]

Step 1:6-(Benzyloxy)-2-bromo-3-(4-(((S)-1-(3-fluoropropyl) pyrrolidin-3-yl)oxy)phenoxy)benzo[b]thiophene 1-oxide

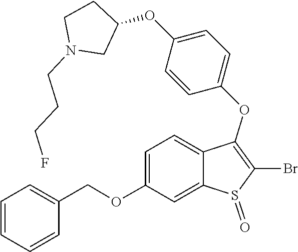 [SMILES: O=S1C(Br)=C(Oc2ccc(O[C@H]3CCN(CCCF)C3)cc2)c2ccc(OCc3ccccc3)cc21]

Step 1 of Example 3 was prepared following a similar procedure to that of step 1 of Example 2 from 6-(benzyloxy)-2,3-dibromobenzo[b]thiophene 1-oxide (Intermediate 2) and(S)-4-((1-(3-fluoropropyl) pyrrolidin-3-yl)oxy) phenol (prepared according to WO2020037251) to give 3.4 g (79%) of 6-(benzyloxy)-2-bromo-3-(4-(((S)-1-(3-fluoropropyl)pyrrolidin-3-yl)oxy)phenoxy)benzo[b]thiophene 1-oxide.

LC/MS (m/z, MH+): 572

### Step 2: (S)-3-(4-((6-(Benzyloxy)-2-bromobenzo[b]thiophen-3-yl)oxy)phenoxy)-1-(3-fluoropropyl)pyrrolidine

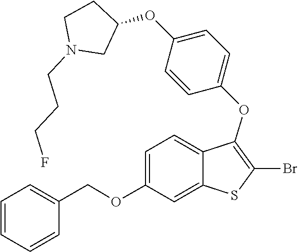 [SMILES: FCCCN1CC[C@H](Oc2ccc(Oc3c(Br)sc4cc(OCc5ccccc5)ccc34)cc2)C1]

Step 2 of Example 3 was prepared following a similar procedure to that of step 2 of Example 2 from 6-(benzyloxy)-2-bromo-3-(4-(((S)-1-(3-fluoropropyl)pyrrolidin-3-yl)oxy)phenoxy)benzo[b]thiophene 1-oxide to give 4.2 g (86%) of (S)-3-(4-((6-(benzyloxy)-2-bromobenzo[b]thiophen-3-yl)oxy)phenoxy)-1-(3-fluoropropyl)pyrrolidine as a white solid.

LC/MS (m/z, MH+): 554

### Step 3: (S)-3-(4-((6-(Benzyloxy)-2-(2-(1,1-difluoroethyl)-4-fluorophenyl)benzo[b]thiophen-3-yl)oxy)phenoxy)-1-(3-fluoropropyl)pyrrolidine

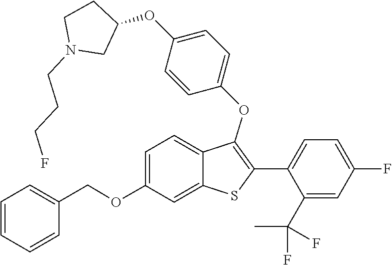 [SMILES: CC(F)(F)c1cc(F)ccc1-c1sc2cc(OCc3ccccc3)ccc2c1Oc1ccc(O[C@H]2CCN(CCCF)C2)cc1]

Step 3 of Example 3 was prepared following a similar procedure to that of step 3 of Example 2 from methyl 2-(2-(1,1-difluoroethyl)-4-fluorophenyl)-3-(4-((1-(3-fluoropropyl)azetidin-3-yl)oxy)phenoxy)benzo[b]thiophene-6-carboxylate and 2-[2-(1,1-difluoroethyl)-4-fluoro-phenyl]-4,4,5,5-tetramethyl-1,3,2-dioxaborolane (prepared according to J. Med. Chem., 2018, 61(7), 2837-2864) to give 390 mg (49%) of (S)-3-(4-((6-(benzyloxy)-2-(2-(1,1-difluoroethyl)-4-fluorophenyl)benzo[b]thiophen-3-yl)oxy)phenoxy)-1-(3-fluoropropyl)pyrrolidine as a white solid.

LC/MS (m/z, MH+): 636

### Step 4: (S)-2-(2-(1,1-Difluoroethyl)-4-fluorophenyl)-3-(4-((1-(3-fluoropropyl)pyrrolidin-3-yl)oxy)phenoxy)benzo[b]thiophen-6-ol

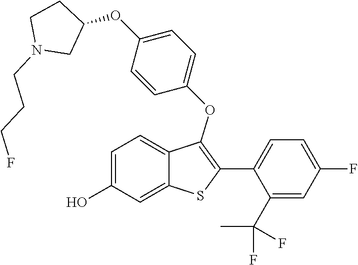 [SMILES: CC(F)(F)c1cc(F)ccc1-c1sc2cc(O)ccc2c1Oc1ccc(O[C@H]2CCN(CCCF)C2)cc1]

Step 4 of Example 3 was prepared following a similar procedure to that of step 4 of Example 2 from methyl 2-(2-(1,1-difluoroethyl)-4-fluorophenyl)-3-(4-((1-(3-fluoropropyl)azetidin-3-yl)oxy)phenoxy)benzo[b]thiophene-6-carboxylate to give 100 mg (51%) of (S)-2-(2-(1,1-difluoroethyl)-4-fluorophenyl)-3-(4-((1-(3-fluoropropyl)pyrrolidin-3-yl)oxy)phenoxy)benzo[b]thiophen-6-ol as a white solid.

LC/MS (m/z, MH+): 546

### Step 5: (S)-2-(2-(1,1-Difluoroethyl)-4-fluorophenyl)-3-(4-((1-(3-fluoropropyl)pyrrolidin-3-yl)oxy)phenoxy)benzo[b]thiophen-6-yl trifluoromethanesulfonate

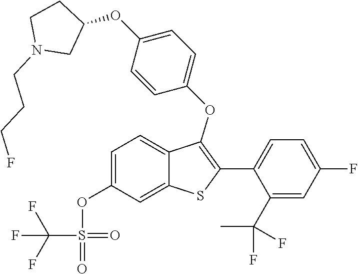 [SMILES: CC(F)(F)c1cc(F)ccc1-c1sc2cc(OS(=O)(=O)C(F)(F)F)ccc2c1Oc1ccc(O[C@H]2CCN(CCCF)C2)cc1]

Step 5 of Example 3 was prepared following a similar procedure to that of step 5 of Example 2 from (S)-2-(2-(1,1-difluoroethyl)-4-fluorophenyl)-3-(4-((1-(3-fluoropropyl)pyrrolidin-3-yl)oxy)phenoxy)benzo[b]thiophen-6-ol to give 130 mg (70%) of (S)-2-(2-(1,1-difluoroethyl)-4-fluorophenyl)-3-(4-((1-(3-fluoropropyl)pyrrolidin-3-yl)oxy)phenoxy)benzo[b]thiophen-6-yl trifluoromethanesulfonate as a white solid.

LC/MS (m/z, MH+): 678

### Step 6: Methyl (S)-2-(2-(1,1-difluoroethyl)-4-fluorophenyl)-3-(4-((1-(3-fluoropropyl)pyrrolidin-3-yl)oxy)phenoxy)benzo[b]thiophene-6-carboxylate

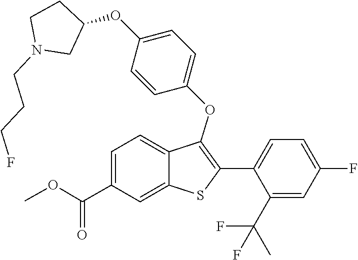 [SMILES: COC(=O)c1ccc2c(Oc3ccc(O[C@H]4CCN(CCCF)C4)cc3)c(-c3ccc(F)cc3C(C)(F)F)sc2c1]

Step 6 of Example 3 was prepared following a similar procedure to that of step 6 of Example 2 from (S)-2-(2-(1,1-difluoroethyl)-4-fluorophenyl)-3-(4-((1-(3-fluoropropyl)pyrrolidin-3-yl)oxy)phenoxy)benzo[b]thiophen-6-yl trifluoromethanesulfonate to give 100 mg (89%) of methyl (S)-2-(2-(1,1-difluoroethyl)-4-fluorophenyl)-3-(4-((1-(3-fluoropropyl)pyrrolidin-3-yl)oxy)phenoxy)benzo[b]thiophene-6-carboxylate as a white solid.

LC/MS (m/z, MH+): 588

### Step 7: 2-(2-(1,1-Difluoroethyl)-4-fluorophenyl)-3-(4-((1-(3-fluoropropyl)azetidin-3-yl)oxy)phenoxy)benzo[b]thiophene-6-carboxylic acid

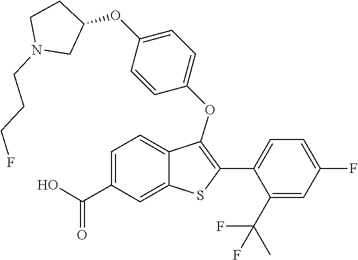 [SMILES: CC(F)(F)c1cc(F)ccc1-c1sc2cc(C(=O)O)ccc2c1Oc1ccc(O[C@H]2CCN(CCCF)C2)cc1]

Step 7 of Example 3 was prepared following a similar procedure to that of step 4 of Example 1 from methyl (S)-2-(2-(1,1-difluoroethyl)-4-fluorophenyl)-3-(4-((1-(3-fluoropropyl)pyrrolidin-3-yl)oxy)phenoxy)benzo[b]thiophene-6-carboxylate to give 81 mg (83%) of 2-(2-(1,1-difluoroethyl)-4-fluorophenyl)-3-(4-((1-(3-fluoropropyl)azetidin-3-yl)oxy)phenoxy)benzo[b]thiophene-6-carboxylic acid as a white solid.

The compounds according to table 1 above were subjected to pharmacological tests for determining their degradation effects on estrogen receptors.

### Test: Estrogen Receptor Degradation Activity

Said test involves measuring the in vitro degradation activity of the compounds of the table 1.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at RT and then washed twice with PBS. Then, 50 μL of LI-COR blocking buffer containing 0.1% Triton was added to plate for 30 minutes at RT. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or ICso) in nM.

The % of ERα levels decrease were determined as follows: % inhibition=100*(1−(sample−fulvestrant:DMSO−fulvestrant)).

The Table 2 below indicates the estrogen receptor degradation activity results for the compounds of table 1 tested at 0.3 μM, and demonstrates that said compounds have a significant degradation activity on estrogen receptors.

TABLE 2

| | Degradation | % Degradation |
|---|---|---|
| Compound No. | IC₅₀ (nM) | At 0.3 μM |
| 1 | 2 | 92 |
| 2 | 0.9 | 93 |
| 3 | 5 | 85 |

It is therefore apparent that the tested compounds have degradation activities for estrogen receptors, with IC50 less than 1 μM and with degradation levels greater than 50%. The compounds of formula (I) can therefore be used for preparing medicaments, especially medicaments which are degraders of estrogen receptors.

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | | |
|---|---|---|
| Compound of formula (I) | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 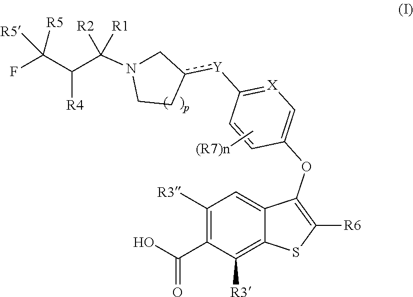
wherein:
R1 and R2 independently represent a hydrogen atom or a deuterium atom;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
Y represents —CH₂—, —CH═, —CR8=, —O— or —NH—, wherein R8 represents a fluorine atom or a (C₁-C₃)alkyl group; [symbol]
represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
n is 0, 1 or 2; and
R6 represents a (C₆-C₁₀)aryl group or a 5- or 6-membered heteroaryl group,
said (C₆-C₁₀)aryl group or a 5- or 6-membered monocyclic heteroaryl group being optionally substituted with 1 to 3 groups selected from —Z—R6a and R6a;
wherein
Z is methylene;
R6a is selected from
a —OH group,
a (C₁-C₆)alkyl group optionally substituted by a cyano group or a —OH group,
a (C₁-C₆)alkylene group,
a halogen atom,
a nitro group,
a cyano group,
a (C₁-C₆)fluoroalkyl group,
a (C₁-C₆)fluoroalkoxy group,
a (C₃-C₆)cycloalkyl group,
a (C₁-C₆)alkoxy group,
a —SF₅ group,
a trifluoromethylsulfonyl group,
a (C₁-C₄)alkylthio group;
a (C₁-C₄)fluoroalkylthio group,
a (C₁-C₄)alkylsulfonyl group
a —NR9R10 group,
a —C(O)R9 group,
a (C₃-C₇)cycloalkyl group, and
a 4-7 membered saturated, unsaturated or partially saturated ring containing one to four heteroatoms or groups selected from O, NH, C(O) and S(O)₀₋₂;
wherein R9 and R10 are independently selected from a hydrogen atom and a (C₁-C₄)alkyl group; or R9 and R10 together with the nitrogen to which they are both attached form a 4 to 7 member saturated ring containing one other heteroatom or group selected from O, NH, and S(O)₀₋₂; wherein said 4-7 member ring can be unsubstituted or substituted with a (C₁-C₄)alkyl group.

2. The compound of formula (I) according to claim 1, or a pharmaceutically acceptable salt thereof, characterized in that R1 and R2 are a hydrogen atom.

3. The compound of formula (I) according to claim 1 or 2, or a pharmaceutically acceptable salt thereof, characterized in that R3′ and R3″ both represent a hydrogen atom.

4. The compound of formula (I) according to any one of claims 1 to 3, or a pharmaceutically acceptable salt thereof, characterized in that R4 represent a hydrogen atom.

5. The compound of formula (I) according to any one of claims 1 to 4, or a pharmaceutically acceptable salt thereof, characterized in that R5 and R5′ both represent a hydrogen atom.

6. The compound of formula (I) according to any one of claims 1 to 5, or a pharmaceutically acceptable salt thereof, characterized in that Y is —O— or —NH—.

7. The compound of formula (I) according to any one of claims 1 to 6, or a pharmaceutically acceptable salt thereof, characterized in that [symbol] represents a single bond.

8. The compound of formula (I) according to any one of claims 1 to 7, or a pharmaceutically acceptable salt thereof, characterized in that n is 0.

9. The compound of formula (I) according to any one of claims 1 to 8, or a pharmaceutically acceptable salt thereof, characterized in that X represents —CH═.

10. The compound of formula (I) according to any one of claims 1 to 9, or a pharmaceutically acceptable salt thereof, characterized in that R6 represents a phenyl group optionally substituted by 1, 2, 3 or 4 groups selected from a halogen atom, and a (C₁-C₆)fluoroalkyl group, more particularly optionally substituted by 2 or 3 groups selected from a halogen, for example a fluorine atom and a (C₁-C₄)fluoroalkyl group and even more particularly by one fluorine atom and by one difluoroethyl group, for example a 1,1-difluoroethyl group.

11. The compound of formula (I) according to any one of claims 1 to 10, or a pharmaceutically acceptable salt thereof, characterized in that X represents —CH═, and R6 represents a phenyl group optionally substituted by 1, 2, 3 or 4 groups selected from a halogen atom, and a (C₁-C₆)fluoroalkyl group, more particularly optionally substituted by 2 or 3 groups selected from a halogen, for example a fluorine atom and a (C₁-C₄)fluoroalkyl group and even more particularly by one fluorine atom and by one difluoroethyl group, for example a 1,1-difluoroethyl group.

12. The compound of formula (I) according to anyone of claims 1 to 9, or a pharmaceutically acceptable salt thereof, in particular hydrochloride salt thereof, characterized in that said compound is selected from the following compounds:
2-(2-(1,1-difluoroethyl)-4-fluorophenyl)-3-(4-((1-(3-fluoropropyl)azetidin-3-yl)amino)phenoxy)benzo[b]thiophene-6-carboxylic acid, hydrochloride (1),
2-(2-(1,1-difluoroethyl)-4-fluorophenyl)-3-(4-((1-(3-fluoropropyl)azetidin-3-yl)oxy)phenoxy)benzo[b]thiophene-6-carboxylic acid (2), and
(S)-2-(2-(1,1-difluoroethyl)-4-fluorophenyl)-3-(4-((1-(3-fluoropropyl)pyrrolidin-3-yl)oxy)phenoxy)benzo[b]thiophene-6-carboxylic acid (3).

13. A process for preparing a compound of formula (I) as described in anyone of claims 1 to 12, wherein a compound of formula 1M 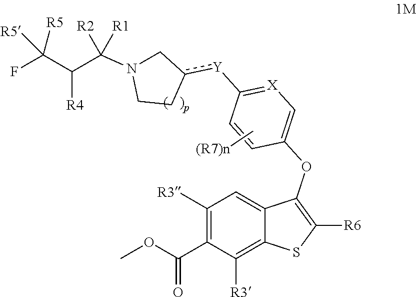
wherein R1, R2, R3′, R3″, R4, R5, R5′, R6, R7, [symbol] n, p, X and Y are defined in any of claims 1 to 11 is converted to compound of formula (I), in presence of a source of hydroxide ions, such as NaOH or LiOH in solution in methanol or THF, said step being optionally preceded by a step of obtaining compound 1M, wherein a compound of formula 1L 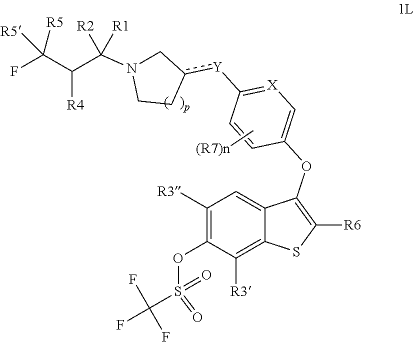
wherein R1, R2, R3′, R3″, R4, R5, R5′, R6, R7, [symbol] , n, p, X and Y are defined above, is converted to compound 1M by carbonylation with carbon monoxide, in solution in DMF and MeOH, in the presence of a palladium catalyst.

14. A process for preparing a compound of formula (I) as described in anyone of claims 1 to 12, wherein a compound of formula 1M 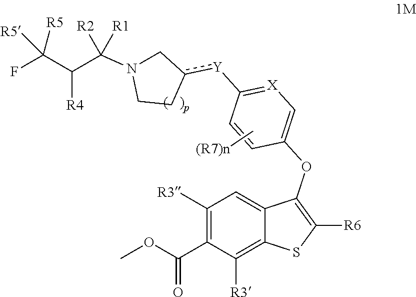
wherein R1, R2, R3′, R3″, R4, R5, R5′, R6, R7, [symbol] n, p, X and Y are defined in any of claims 1 to 11 is converted to compound of formula (I), in presence of a source of hydroxide ions, such as NaOH or LiOH in solution in methanol or THF, said step being optionally preceded by a step of obtaining compound 1M, wherein a compound of formula 2B 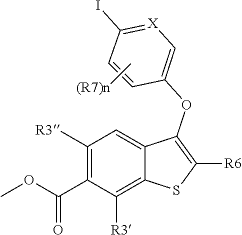
wherein R3′, R3″, R6, R7, n and X are defined above, is coupled with one of compounds 1G in coupling reaction conditions. 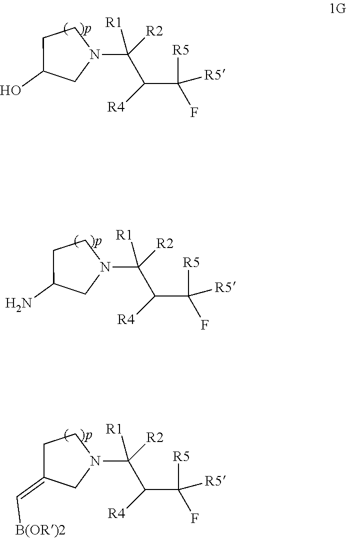
wherein R1, R2, R3′, R3″, R4, R5, R5′ and p are defined above.

15. A process for preparing a compound of formula (I) as described in anyone of claims 1 to 12, wherein a compound of formula 1M 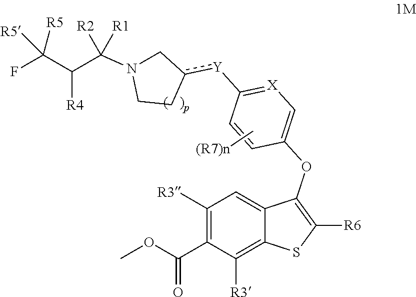
wherein R1, R2, R3′, R3″, R4, R5, R5′, R6, R7, [symbol] n, p, X and Y are defined in any of claims 1 to 11 is converted to compound of formula (I), in presence of a source of hydroxide ions, such as NaOH or LiOH in solution in methanol or THF, said step being optionally preceded by a step of obtaining compound 1M, wherein a compound of formula 2A 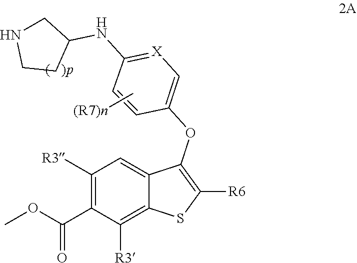
wherein R3′, R3″, R6, R7, n, p and X are defined above, is treated with compound 1Q 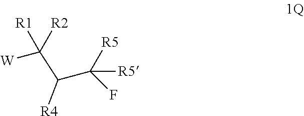
wherein W is Cl, Br or I or OSO₂R with R=CH₃, PhMe, CF₃ or CF₂CF₂CF₂CF₃ and R1, R2, R3′, R3″, R4, R5, R5′ and p are as defined above, in presence of a base such as potassium carbonate in dichloromethane as a solvent.

16. Compounds selected from compounds 1M, 1H, 1L and 1X 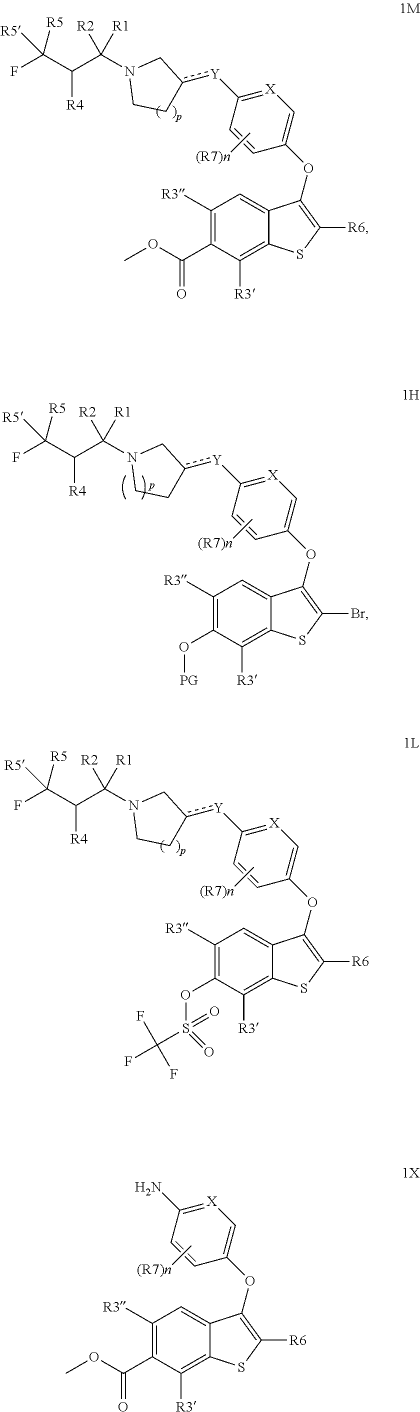
wherein R1, R2, R3, R3′, R3″, R4, R5, R5′, R6, R7, n, p, [symbol] X and Y are defined in any of claims 1 to 11 and PG is a protecting group such as pivaloyl, benzyl, para-methoxy-benzyl or 2,4-dimethoxy-benzyl.

17. A medicament, characterized in that it comprises a compound of formula (I) according to any of claims 1 to 12, or a pharmaceutically acceptable salt thereof.

18. A pharmaceutical composition, characterized in that it comprises a compound of formula (I) according to any of claims 1 to 12, or a pharmaceutically acceptable salt thereof, and at least one pharmaceutically acceptable excipient.

19. A compound of formula (I) according to any of claims 1 to 12, or a pharmaceutically acceptable salt thereof, for use as an inhibitor and degrader of estrogen receptors.

20. A compound of formula (I) according to any of claims 1 to 12, or a pharmaceutically acceptable salt thereof, for use in the treatment of ovulatory dysfunction, cancer, endometriosis, osteoporosis, benign prostatic hypertrophy or inflammation.

21. A compound of formula (I) for use according to claim 20, or a pharmaceutically acceptable salt thereof, for use in the treatment of cancer.
